(19) United States
(12) Patent Application Publication
(10) Pub. No.: US 2024/0042003 A1
(43) Pub. Date: Feb. 8, 2024

(54) CROSS-PROTECTIVE ANTIGENS FOR VACCINATION

(71) Applicant: THE WEST VIRGINIA UNIVERSITY BOARD OF GOVERNORS ON BEHALF OF WEST VIRGINIA UNIVERSITY, Morgantown, WV (US)

(72) Inventors: Mariette BARBIER, Morgantown, WV (US); Fredrick Heath DAMRON, Morgantown, WV (US); Catherine B. BLACKWOOD, Morgantown, WV (US)

(21) Appl. No.: 18/483,888

(22) Filed: Oct. 10, 2023

Related U.S. Application Data
(63) Continuation of application No. PCT/US2022/023999, filed on Apr. 8, 2022.
(60) Provisional application No. 63/172,259, filed on Apr. 8, 2021.

(51) Int. Cl. A61K 39/02; A61K 39/39; A61K 39/104

(52) U.S. Cl. CPC A61K 39/099; A61K 39/39; A61K 39/104; A61K 2039/10

(57) ABSTRACT

A method of immunizing a mammalian patient against infection by a bacterial pathogen involves administering a pertussis or Pseudomonas antigen to the mammalian patient. The pertussis antigen is least one of a chaperonin protein GroEL from Bordetella pertussis, or a fragment thereof; and an OmpA protein of Bordetella pertussis, or a fragment thereof. The Pseudomonas antigen is least one of a chaperonin protein GroEL from Pseudomonas aeruginosa, or a fragment thereof; and an OprF protein from Pseudomonas aeruginosa, or an OmpA-domain fragment thereof. The bacterial pathogen expresses a protein having at least 45% identity to the pertussis antigen. The bacterial pathogen may be a gram-negative bacteria. The bacterial pathogen may be a bacteria from a genus Escherichia, a genus Enterococcus, a genus Staphylococcus, a genus Klebsiella, a genus Acinetobacter, and a genus Enterobacter.

### CROSS-REFERENCE TO RELATED APPLICATIONS

This application is a continuation application of International Application No. PCT/US22/23999, filed on Apr. 8, 2022. This application claims the benefit of U.S. Provisional Application No. 63/172,259, filed on Apr. 8, 2021. The entire disclosure of each prior application is incorporated by reference herein.

### STATEMENT ACCORDING TO 37 CFR 1.833: SEQUENCE LISTING SUBMITTED AS XML FILE

Pursuant to 37 C.F.R. § 1.833, this application contains a sequence listing, which is contained on an XML file entitled “WVU 3059-US Sequence Listing.xml,” created Oct. 4, 2023, having a size of 23,106 bytes, which is herein incorporated by reference.

### TECHNICAL FIELD

Various embodiments disclosed herein relate generally to immunizing a mammalian patient against infection by a bacterial pathogen, including administering a pertussis antigen to the mammalian patient, where the bacterial pathogen may be from a genus other than Bordetella. Some embodiments disclosed herein relate to immunizing a mammalian patient against infection by a bacterial pathogen, including administering a Pseudomonas antigen to the mammalian patient, where the bacterial pathogen may be from a genus other than Pseudomonas, e.g., B. pertussis.

### BACKGROUND

Antibiotic-resistant bacterial infections represent a significant burden in the United States and globally, accounting for more than 2.8 million infections and 35,000 deaths yearly in the U.S. alone. As a result, the threat of antibiotic resistant infections continues to drive the development of preventative measures, such as vaccines. Whole cell vaccines are designed to train the immune system to mount a response against a pathogen of interest. Antigens contained in the vaccines are processed, presented by antigen-presenting cells, and trigger the activation of an adaptive immune response. This pathogen-specific immune response can then be recalled during a potential exposure to the live pathogen.

While the protection of whole cell vaccines is typically studied in the context of protection from the pathogen contained in the formulation, nonspecific effects of whole cell vaccines have also been observed. These nonspecific effects can be mediated by both innate and adaptive immune systems in response to vaccination. In some instances, vaccination can protect against other pathogens through the production of cross-reactive antibodies that bind closely related antigens in other organisms. For example, some anti-flu antibodies can protect against heterologous strains of the virus, and exposure to flaviviruses prevents subsequent infection by other flaviviruses.

In addition to protection mediated by cross-reactive antibodies, it is now well-established that some vaccines are able to induce trained innate immunity that leads to epigenetic reprogramming of innate immune cells, including monocytes and macrophages, allowing for a quicker response to exposure to a different pathogen. For example, the Bacille Calmette-Guérin (BCG) vaccine for Mycobacterium tuberculosis used in countries where TB is still prevalent, modulates the innate immune response and induces protection against non-mycobacterium species and some types of cancers.

A growing body of evidence supports that whole cell pertussis vaccines (denoted here as Bp-WCV) also provide non-specific protection against other pathogens. Bp-WCV protects against Bordetella pertussis, the causative agent of whooping cough. Co-administration of the Diphtheria, Tetanus, and Pertussis (DTP) vaccine with BCG is associated with increased efficacy of the BCG vaccine. Furthermore, the live-attenuated B. pertussis vaccine BPZE1, currently in a clinical phase of development, is known to induce a short-term cross-protective response against S. pneumoniae.

### SUMMARY

In light of the present need for improved vaccines, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a method of immunizing a mammalian patient against infection by a bacterial pathogen, by administering a pertussis antigen to the mammalian patient. The pertussis antigen may be least one of a chaperonin protein GroEL from Bordetella pertussis, or a fragment thereof; an OmpA protein of Bordetella pertussis; a fragment thereof; and a combination thereof. The bacterial pathogen may express a protein having at least 45%, at least 50%, at least 60%, at least 70%, at least 75%, at least 80%, at least 90%, or at least 95% identity to the pertussis antigen. In various embodiments, the bacterial pathogen may be a gram-negative bacterial pathogen. The bacterial pathogen may be a bacteria from a genus Bordetella, a genus Pseudomonas, a genus Escherichia, a genus Enterococcus, a genus Staphylococcus, a genus Klebsiella, a genus Acinetobacter, and a genus Enterobacter. The bacterial pathogen may be from a species selected from the group consisting of Bordetella pertussis, Bordetella bronchiseptica, Enterococcus faecium, Staphylococcus aureus, Escherichia coli, Klebsiella pneumoniae, Acinetobacter baumannii, Pseudomonas aeruginosa, and an Enterobacter species. The bacterial pathogen may be an ESKAPE pathogen selected from the group consisting of Enterococcus faecium, Staphylococcus aureus, Klebsiella pneumoniae, Acinetobacter baumannii, Pseudomonas aeruginosa, and an Enterobacter species.

Various embodiments disclosed herein relate to a method of immunizing a mammalian patient against infection by a bacterial pathogen, by administering a pertussis antigen to the mammalian patient, where the pertussis antigen is conjugated to a carrier protein or an amino acid tag. The pertussis antigen may be least one of a chaperonin protein GroEL from Bordetella pertussis, or a fragment thereof; an OmpA protein of Bordetella pertussis; a fragment thereof; and a combination thereof.

The pertussis antigen may be the chaperonin protein GroEL from Bordetella pertussis, and the bacterial pathogen may express a protein having at least 60%, at least 75%, at least 80%, at least 90%, or at least 95% identity to the chaperonin protein GroEL from Bordetella pertussis.

In various embodiments, the pertussis antigen may be a fragment of the Bordetella pertussis chaperonin protein GroEL having at least 80%, 85%, 90%, 95%, 99%, or 100% identity to:
a sequence having amino acid residues 30 to 40 of SEQ ID NO: 16,
a sequence having amino acid residues 50 to 98 of SEQ ID NO: 16,
a sequence having amino acid residues 168 to 178 of SEQ ID NO: 16,
a sequence having amino acid residues 189 to 204 of SEQ ID NO: 16,
a sequence having amino acid residues 251 to 304 of SEQ ID NO: 16,
a sequence having amino acid residues 326 to 349 of SEQ ID NO: 16;
a sequence having amino acid residues 381 to 419 of SEQ ID NO: 16.

The bacterial pathogen may express a protein having at least 60%, 70%, 80%, 85%, 90%, or 95% identity to the chaperonin protein GroEL from Bordetella pertussis.

The pertussis antigen may be a fragment of the OmpA protein of Bordetella pertussis including SEQ ID NO: 9, and the bacterial pathogen may express a protein having a sequence with at least 50%, at least 60%, at least 75%, at least 80%, at least 90%, or at least 95% identity to SEQ ID NO: 9. The OmpA protein of Bordetella pertussis is represented by SEQ ID No: 8, and SEQ ID NO: 9 includes amino acid residues 74 to 193 of SEQ ID No: 8.

The pertussis antigen may be a fragment of the OmpA protein of Bordetella pertussis including SEQ ID NO: 12, and the bacterial pathogen may express a protein having a sequence with at least 75%, at least 80%, at least 90%, or at least 95% identity to SEQ ID NO: 12. SEQ ID NO: 12 includes amino acid residues 121 to 186 of SEQ ID No: 8.

The pertussis antigen may be a fragment of the OmpA protein of Bordetella pertussis including SEQ ID NO: 10, and the bacterial pathogen may express a protein having a sequence with at least 85%, at least 90%, or at least 95% identity to SEQ ID NO: 10. SEQ ID NO: 10 includes amino acid residues 121 to 145 of SEQ ID No: 8.

The pertussis antigen may be a fragment of the OmpA protein of Bordetella pertussis including SEQ ID NO: 11, and the bacterial pathogen may express a protein having a sequence with at least 85%, at least 90%, or at least 95% identity to SEQ ID NO: 11. SEQ ID NO: 11 includes amino acid residues 170 to 186 of SEQ ID No: 8.

In various embodiments, the pertussis antigen is administered intranasally, intravenously, intramuscularly, subcutaneously, intradermally, or intraperitoneally. The pertussis antigen may be administered in combination with an adjuvant selected from the group consisting of curdlan and other β-glucans, alum, amorphous aluminum hydroxyphosphate sulfate, aluminum hydroxide, aluminum phosphate, monophosphoryl lipid A, squalene, cytosine phosphoguanine, MF59, AS03, AS04, BECC adjuvants (Bacterial Enzymatic Combinatorial Chemistry), SWE, or combinations thereof.

Various embodiments disclosed herein relate to a method of immunizing a mammalian patient against infection by a bacterial pathogen, including administering a Pseudomonas antigen to the mammalian patient. The Pseudomonas antigen may be least one of a chaperonin protein GroEL from Pseudomonas aeruginosa, or a fragment thereof; an OprF protein from Pseudomonas aeruginosa, or a fragment thereof; and a combination thereof. The bacterial pathogen may express a protein having at least 45%, at least 50%, at least 60%, at least 70%, at least 75%, at least 80%, at least 90%, or at least 95% identity to the Pseudomonas antigen. In various embodiments, the bacterial pathogen may be from a different species than a Pseudomonas species, and may be a bacteria from a genus Bordetella, a genus Escherichia, a genus Enterococcus, a genus Staphylococcus, a genus Klebsiella, a genus Acinetobacter, a genus Enterobacter, or a combination thereof.

The Pseudomonas antigen may be the chaperonin protein GroEL from Pseudomonas aeruginosa, and the bacterial pathogen may express a protein having at least 60%, at least 75%, at least 80%, at least 90%, or at least 95% identity to the chaperonin protein GroEL from Pseudomonas aeruginosa.

The Pseudomonas antigen may be a fragment of the OprF protein of Pseudomonas aeruginosa including an OmpA domain, and the bacterial pathogen may express a protein having a sequence with at least 50%, at least 60%, at least 75%, at least 80%, at least 90%, or at least 95% identity to the OmpA domain.

The Pseudomonas antigen may be a fragment of the OprF protein of Pseudomonas aeruginosa including SEQ ID NO: 3, and the bacterial pathogen may express a protein having a sequence with at least 50%, at least 60%, at least 70%, at least 75%, at least 80%, at least 90%, or at least 95% identity to SEQ ID NO: 3. The OprF protein of Pseudomonas aeruginosa is represented by SEQ ID No: 2, and SEQ ID NO: 3 includes amino acid residues 231 to 350 of SEQ ID No: 2.

The Pseudomonas antigen may be a fragment of the OprF protein of Pseudomonas aeruginosa including SEQ ID NO: 6, and the bacterial pathogen may express a protein having a sequence with at least 75%, at least 80%, at least 90%, or at least 95% identity to SEQ ID NO: 6. SEQ ID NO: 6 includes amino acid residues 278 to 344 of SEQ ID No: 2.

The Pseudomonas antigen may be a fragment of the OprF protein of Pseudomonas aeruginosa including SEQ ID NO: 4, and the bacterial pathogen may express a protein having a sequence with at least 75%, at least 80%, at least 90%, or at least 95% identity to SEQ ID NO: 4. SEQ ID NO: 4 includes amino acid residues 278 to 303 of SEQ ID No: 2.

The Pseudomonas antigen may be a fragment of the OprF protein of Pseudomonas aeruginosa including SEQ ID NO: 5, and the bacterial pathogen may express a protein having a sequence with at least 85%, at least 90%, or at least 95% identity to SEQ ID NO: 5. SEQ ID NO: 5 includes amino acid residues 328 to 344 of SEQ ID No: 2.

In various embodiments, the Pseudomonas antigen is administered intranasally, intravenously, intramuscularly, subcutaneously, intradermally, orally, rectally, or intraperitoneally. The Pseudomonas antigen may be administered in combination with an adjuvant selected from the group consisting of curdlan and other β-glucans, alum, amorphous aluminum hydroxyphosphate sulfate, aluminum hydroxide, aluminum phosphate, monophosphoryl lipid A, squalene, cytosine phosphoguanine, MF59, AS03, AS04, BECC adjuvants (Bacterial Enzymatic Combinatorial Chemistry), SWE, or combinations thereof.

Various embodiments disclosed herein relate to a method of providing an enhanced immune response to infection by a bacterial pathogen, including administering to a mammalian patient a combination of a first pertussis antigen, and a second antigen to the bacterial pathogen. The bacterial pathogen may be selected from the group consisting of bacteria from a genus Pseudomonas, a genus Escherichia, a genus Enterococcus, a genus Staphylococcus, a genus Klebsiella, a genus Acinetobacter, and a genus Enterobacter; and the pertussis antigen may be a chaperonin protein GroEL from Bordetella pertussis; an OmpA protein of Bordetella pertussis; a combination thereof; or a mixture thereof. The second antigen may be, but is not limited to, a chaperonin protein GroEL from Pseudomonas aeruginosa, or a fragment thereof; an OprF protein from Pseudomonas aeruginosa, or a fragment thereof; or a combination thereof.

In various embodiments, the bacterial pathogen may be selected from the group consisting of bacteria from a genus Pseudomonas, a genus Escherichia, a genus Enterococcus, a genus Staphylococcus, a genus Klebsiella, a genus Acinetobacter, and a genus Enterobacter, and an enhanced immune response to infection is provided by administering a combination of:
a pertussis antigen which is a chaperonin protein GroEL from Bordetella pertussis; an OmpA protein of Bordetella pertussis; a fragment thereof, or a combination thereof; and
a second antigen including a protein or protein fragment derived from the bacterial pathogen.

In various embodiments, the combination of the first pertussis antigen and the second antigen may be administered intranasally, intravenously, intramuscularly, subcutaneously, intradermally, orally, rectally, or intraperitoneally. The combination of the first pertussis antigen and the second antigen may be administered in combination with an adjuvant selected from the group consisting of curdlan and other β-glucans, alum, amorphous aluminum hydroxyphosphate sulfate, aluminum hydroxide, aluminum phosphate, monophosphoryl lipid A, squalene, cytosine phosphoguanine, MF59, AS03, AS04, BECC adjuvants (Bacterial Enzymatic Combinatorial Chemistry), SWE, or combinations thereof. The first pertussis antigen and the second antigen may be administered separately or in combination.

Various embodiments disclosed herein relate to therapeutically effective antibodies generated against B. pertussis GroEL having at least 85% identity to SEQ ID NO: 16 or a fragment thereof, where the antibody has cross reactivity against P. aeruginosa bacteria expressing P. aeruginosa GroEL. The fragment of B. pertussis GroEL used to generate the antibody may be:
A B. pertussis GroEL fragment having at least 85% identity to the sequence having amino acid residues 30 to 40 of SEQ ID NO: 16;
A B. pertussis GroEL fragment having at least 85% identity to the sequence having amino acid residues 50 to 98 of SEQ ID NO: 16;
A B. pertussis GroEL fragment having at least 85% identity to the sequence having amino acid residues 168 to 178 of SEQ ID NO: 16;
A B. pertussis GroEL fragment having at least 85% identity to the sequence having amino acid residues 189 to 204 of SEQ ID NO: 16;
A B. pertussis GroEL fragment having at least 85% identity to the sequence having amino acid residues 251 to 304 of SEQ ID NO: 16;
A B. pertussis GroEL fragment having at least 85% identity to the sequence having amino acid residues 326 to 349 of SEQ ID NO: 16; or
A B. pertussis GroEL fragment having at least 85% identity to the sequence having amino acid residues 381 to 419 of SEQ ID NO: 16.

Various embodiments disclosed herein relate to therapeutically effective antibodies generated against P. aeruginosa GroEL having at least 85% identity to SEQ ID NO: 14 or a fragment thereof, where the antibody has cross reactivity against other species of bacteria expressing GroEL. The fragment of P. aeruginosa GroEL used to generate the antibody may be:
A P. aeruginosa GroEL fragment having at least 85% identity to the sequence having amino acid residues 30 to 40 of SEQ ID NO: 14;
A P. aeruginosa GroEL fragment having at least 85% identity to the sequence having amino acid residues 50 to 98 of SEQ ID NO: 14;
A P. aeruginosa GroEL fragment having at least 85% identity to the sequence having amino acid residues 168 to 178 of SEQ ID NO: 14;
A P. aeruginosa GroEL fragment having at least 85% identity to the sequence having amino acid residues 189 to 204 of SEQ ID NO: 14;
A P. aeruginosa GroEL fragment having at least 85% identity to the sequence having amino acid residues 251 to 304 of SEQ ID NO: 14;
A P. aeruginosa GroEL fragment having at least 85% identity to the sequence having amino acid residues 326 to 349 of SEQ ID NO: 14; or
A P. aeruginosa GroEL fragment having at least 85% identity to the sequence having amino acid residues 381 to 419 of SEQ ID NO: 14.

Various embodiments disclosed herein relate to therapeutically effective antibodies generated against B. pertussis OmpA having at least 85% identity to SEQ ID NO: 8 or a fragment thereof, where the antibody has cross reactivity against other species of bacteria. The fragment of B. pertussis OmpA used to generate the antibody may be:
a fragment of the B. pertussis OmpA having at least 85% identity to SEQ ID NO: 9,
a fragment of the B. pertussis OmpA having at least 85% identity to SEQ ID NO: 12,
a fragment of the B. pertussis OmpA having at least 85% identity to SEQ ID NO: 10, or
a fragment of the B. pertussis OmpA having at least 85% identity to SEQ ID NO: 11.

### BRIEF DESCRIPTION OF THE DRAWINGS

In order to better understand various exemplary embodiments, reference is made to the accompanying drawings, wherein:

FIG. 1A shows the alignment between the sequences of B. pertussis GroEL (SEQ ID NO: 16) and P. aeruginosa GroEL (SEQ ID NO: 14).

FIG. 1B shows the alignment between the sequences of B. pertussis GroEL and E. coli GroEL (SEQ ID NO: 17).

FIG. 1C shows a key for determining percentage identity along the length of peptide sequences.

FIG. 1D shows the alignment between the sequence of B. pertussis Tohama I OmpA (SEQ ID NO: 8) and P. aeruginosa PAO1 OprF (SEQ ID NO: 2).

FIG. 2 shows the conformational structure of P. aeruginosa GroEL protein and B. pertussis GroEL proteins.

FIG. 3 shows the structures of the OmpA domain of P. aeruginosa OprF and B. pertussis OmpA.

FIGS. 4A and 4B show bacterial loads in mice vaccinated with P. aeruginosa whole cell vaccine and/or B. pertussis whole cell vaccine.

FIGS. 5A, 5B, 5C, and 5D show bacterial loads in mice vaccinated with P. aeruginosa whole cell vaccine and/or B. pertussis OmpA whole cell vaccine.

FIGS. 6A and 6B show the impact of the protective response induced by P. aeruginosa whole cell vaccine, B. pertussis OmpA whole cell vaccine, and the adjuvant alum on respiratory function, e.g., tidal volume of breath (TVb) and inspiration time (Ti).

FIGS. 7A and 7B show that vaccination of mice using either P. aeruginosa whole cell vaccine or B. pertussis whole cell vaccine induced significant levels of anti-P. aeruginosa IgG in serum.

FIGS. 8, 9, and 10 show that immunoblotting with serum from B. pertussis whole cell vaccine-vaccinated mice and each of P. aeruginosa lysate, E. coli GroEL protein, and P. aeruginosa OprF protein demonstrates that antibodies to B. pertussis exhibit cross reactivity with proteins from other bacterial species.

FIG. 11 shows that antigens from B. pertussis may provide protection against clinical P. aeruginosa isolates.

FIG. 12 shows western blot analysis of IgG binding to clinical isolates, indicating that antibodies present in B. pertussis whole cell vaccine-vaccinated serum showed binding to at least two antigens in each clinical isolate, with molecular weights consistent with P. aeruginosa OprF and P. aeruginosa GroEL.

FIG. 13 shows alignment between GroEL of B. pertussis (SEQ ID NO: 16), P. aeruginosa (SEQ ID NO: 14), and E. coli (SEQ ID NO: 17), where conserved sequences of interest are highlighted in bold text.

FIG. 14 shows survival of mice vaccinated with recombinant B. pertussis OmpA, a whole cell P. aeruginosa vaccine (PaWCV), or a whole cell B. pertussis vaccine (BpWCV) formulated with alum, and challenged with a lethal dose of P. aeruginosa.

FIG. 15A shows rectal temperature of mice vaccinated with alum, and challenged with a lethal dose of P. aeruginosa

FIG. 15B shows rectal temperature of mice vaccinated with a whole cell P. aeruginosa vaccine (PaWCV), and challenged with a lethal dose of P. aeruginosa

FIG. 15C shows rectal temperature of mice vaccinated with a whole cell B. pertussis vaccine (BpWCV) formulated with alum, and challenged with a lethal dose of P. aeruginosa

FIG. 15D shows rectal temperature of mice vaccinated with recombinant B. pertussis OmpA, and challenged with a lethal dose of P. aeruginosa

### DESCRIPTION OF THE SEQUENCE LISTING

This application incorporates a sequence listing, including the following sequences:
SEQ ID NO: 1, which is the DNA sequence encoding the OprF protein of Pseudomonas aeruginosa.
SEQ ID NO: 2, which is the sequence of the OprF protein of Pseudomonas aeruginosa.
SEQ ID NO: 3, which is a fragment of the OprF protein of Pseudomonas aeruginosa including an OmpA domain, including amino acid residues 231 to 350 of SEQ ID No: 2.
SEQ ID NO: 4, which is a fragment of the OprF protein of Pseudomonas aeruginosa including amino acid residues 278 to 303 of SEQ ID No: 2.
SEQ ID NO: 5, which is a fragment of the OprF protein of Pseudomonas aeruginosa including amino acid residues 328 to 344 of SEQ ID No: 2.
SEQ ID NO: 6, which is a fragment of the OprF protein of Pseudomonas aeruginosa including amino acid residues 278 to 344 of SEQ ID No: 2.
SEQ ID NO: 7, which is the DNA sequence encoding the OmpA protein of Bordetella pertussis.
SEQ ID NO: 8, which is the sequence of the OmpA protein of Bordetella pertussis.
SEQ ID NO: 9, which is a fragment of the OmpA protein of Bordetella pertussis including amino acid residues 74 to 193 of SEQ ID No: 8.
SEQ ID NO: 10, which is a fragment of the OmpA protein of Bordetella pertussis including amino acid residues 121 to 145 of SEQ ID No: 8.
SEQ ID NO: 11, which is a fragment of the OmpA protein of Bordetella pertussis including amino acid residues 170 to 186 of SEQ ID No: 8.
SEQ ID NO: 12, which is a fragment of the OmpA protein of Bordetella pertussis including amino acid residues 121 to 186 of SEQ ID No: 8.
SEQ ID NO: 13, which is the DNA sequence encoding the GroEL protein of Pseudomonas aeruginosa.
SEQ ID NO: 14, which is the sequence of the GroEL protein of Pseudomonas aeruginosa.
SEQ ID NO: 15, which is the DNA sequence encoding the GroEL protein of Bordetella pertussis.
SEQ ID NO: 16, which is the sequence of the GroEL protein of Bordetella pertussis.
SEQ ID NO: 17, which is the sequence of the GroEL protein of Escherichia coli.

### DETAILED DESCRIPTION

Referring now to the drawings, broad aspects of various embodiments are disclosed herein.

The present disclosure relates to beneficial non-specific effects of a Bp-WCV and Bordetella pertussis antigenic peptides against the antibiotic-resistant bacterium Pseudomonas aeruginosa. This pathogen is a major causative agent of antibiotic-resistant infections and has no vaccine approved for human use. The B-cell mediated immune response is essential for protection during vaccination with a P. aeruginosa whole cell vaccine (Pa-WCV). Thus, methodologies to improve the B-cell mediated immune response to P. aeruginosa vaccination are needed to increase vaccine efficacy. As discussed herein, vaccination with Bp-WCV, or proteins from Bordetella pertussis, may enhance the immune response to P. aeruginosa, in a manner similar to BCG.

B. pertussis whole cell vaccination may act as an adjuvant when formulated with other vaccines and protects against other bacterial pathogens. Bp-WCV may have adjuvant-like properties when administered in combination with Pa-WCV, and enhance protection against P. aeruginosa challenge.

The protective role of a Bp-WCV against the Gram-negative pathogen P. aeruginosa is described herein. Bp-WCV induces a protective adaptive immune response against P. aeruginosa in CD-1, C57BL/6, and transgenic β-ENaC mice. Bp-WCV may induce production of anti-P. aeruginosa IgG antibodies, and that the anti-P. aeruginosa IgG antibodies bind the P. aeruginosa proteins GroEL and OprF. These proteins have homologs in Bordetella pertussis that may induce cross-protection between B. pertussis and P. aeruginosa. These antigens are conserved in clinical isolates of P. aeruginosa, as was antibody cross-reactivity.

The antigens identified in this study, GroEL and OprF, are known to be immunogenic and antigenic proteins of P. aeruginosa. GroEL, which works in conjunction with the protein GroES to form a well-characterized barrel-shaped chaperonin, is a protein having 548 amino acid residues, which is conserved amongst many bacterial species. This bacterial protein is highly abundant and immunogenic. GroEL is highly conserved amongst Gram-negative bacteria of clinical relevance. In addition, it is known that GroEL is similar to mitochondrial heat shock protein Hsp60 (51.15% identity of Hsp60 to P. aeruginosa GroEL).

While this characteristic might be an advantage associated with cross-reactivity and potential protection against other bacterial species, caution is advisable when using GroEL as a potential candidate vaccine antigen in humans, due to potential undesired cross-reactivity. For example, an increase in anti-P. aeruginosa GroEL antibody titers are observed in cystic fibrosis proceeding the onset of diabetes, and is hypothesized to play a role in the mitochondrial stress associated with diabetes. If GroEL is a protective antigen for P. aeruginosa, it may be useful to further analyze the epitopes recognized by the Bp-WCV induced cross-reactive antibodies to identify specie-specific epitopes.

The other cross-reactive antigen identified, OprF, is a highly abundant outer membrane porin. OprF also plays significant roles in pathogenesis of P. aeruginosa. OprF is capable of transporting ions and low molecular-mass carbohydrates, such as sodium chloride, glucose, and sucrose. OprF is vital in maintenance of the cell membrane and regulation of cell morphology, quorum sensing, and virulence factors including adhesion to eukaryotic cells, biofilm production, and type 3 secretion system. Deletion or functional knockout of the OprF gene in Pseudomonas aeruginosa leads to decreased virulence in several infection models, including Caenorhabditis elegans, cell culture, and mouse models. OprF is also overexpressed in anaerobic conditions, such as those mimicking CF-like lung phenotypes. Various vaccines containing OprF have been pre-clinically and clinically tested for efficacy. One study found that two peptides, linear epitopes from the OmpA domain, conjugated to the carrier protein KLH were able to induce a protective response in mice. A recombinant OprF-OprI vaccine was tested in humans, and induced strong IgG and IgA antibody response.

P. aeruginosa OprF contains two domains, an N-terminal membrane embedded 8-fold beta-barrel porin, and a C-terminal globular domain made of homo-4-mer known as the OmpA domain. While prior studies focused on the beta-barrel domain of OprF, the structure and sequence homology data obtained in this study seem to indicate that cross-reactivity is potentially mediated via the globular OmpA domain of OprF. OmpA-family proteins are produced by a wide variety of pathogenic and nonpathogenic bacteria, including Haemophilus influenzae, Klebsiella pneumoniae, and Chlamydia trachomatis, but not all species produce both domains found in the P. aeruginosa protein.

Notably, there is 100% conservation of the OprF amino acid sequence in clinical isolates of P. aeruginosa, suggesting that it could be a viable antigen for protection against a variety of strains of the pathogen. In addition, P. aeruginosa produces several OmpA family proteins that could likely be used for vaccination against this bacterium. By examining the conserved epitopes of OmpA domains, it may be possible to target multiple protein antigens simultaneously.

The B. pertussis GroEL and OmpA proteins, present in Bp-WCV, may serve as cross reactive antigens against P. aeruginosa. The non-specific effects of B. pertussis whole cell vaccination may thus provide protection against the respiratory pathogens P. aeruginosa. Since the OmpA domain of OprF and the GroEL sequence are highly conserved in a range of bacterial genera, including bacteria from the genera Escherichia, Enterococcus, Staphylococcus, Klebsiella, Acinetobacter, Enterobacter, Bordetella, and Pseudomonas, the B. pertussis GroEL and OmpA proteins may serve as cross reactive antigens against a range of bacterial pathogens, including the drug-resistant ESKAPE pathogens, i.e., Enterococcus faecium, Staphylococcus aureus, Klebsiella pneumoniae, Acinetobacter baumannii, Pseudomonas aeruginosa, and Enterobacter spp. Similarly, antigens derived from P. aeruginosa GroEL and OprF proteins, or fragments thereof, may serve as cross reactive antigens against B. pertussis bacteria and ESKAPE pathogens. B. pertussis GroEL may also serve to induce an immune response against bacterial pathogen expressing a heat shock protein having at least 50% sequence identity to B. pertussis GroEL.

The data presented here suggests that acellular pertussis vaccines do not provide protection against P. aeruginosa, as the B. pertussis GroEL and OmpA antigenic proteins may not be present in significant quantities in acellular pertussis vaccines. However, studies focused on vaccination using the B. pertussis vaccines show that the GroEL and OmpA proteins may induce protective immunity against P. aeruginosa.

TABLE 1

| | GroEL | % Identity to PAO1 |
|---|---|---|
| ESKAPE Pathogen | Homolog | GroEL AA Sequence |
| Enterococcus | Yes | 63.43 |
| Staphylococcus aureus | Yes | 58.47 |
| Klebsiella pneumonia | Yes | 80.07 |
| Acinetobacter baumanii | Yes | 77.17 |
| Gammaproteobacter | Yes | 91.51 |

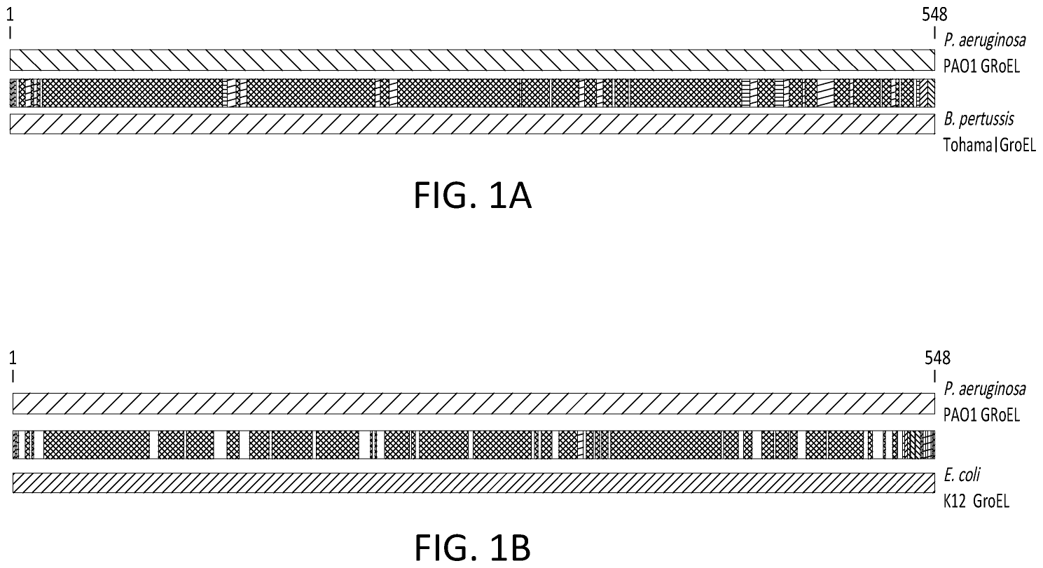

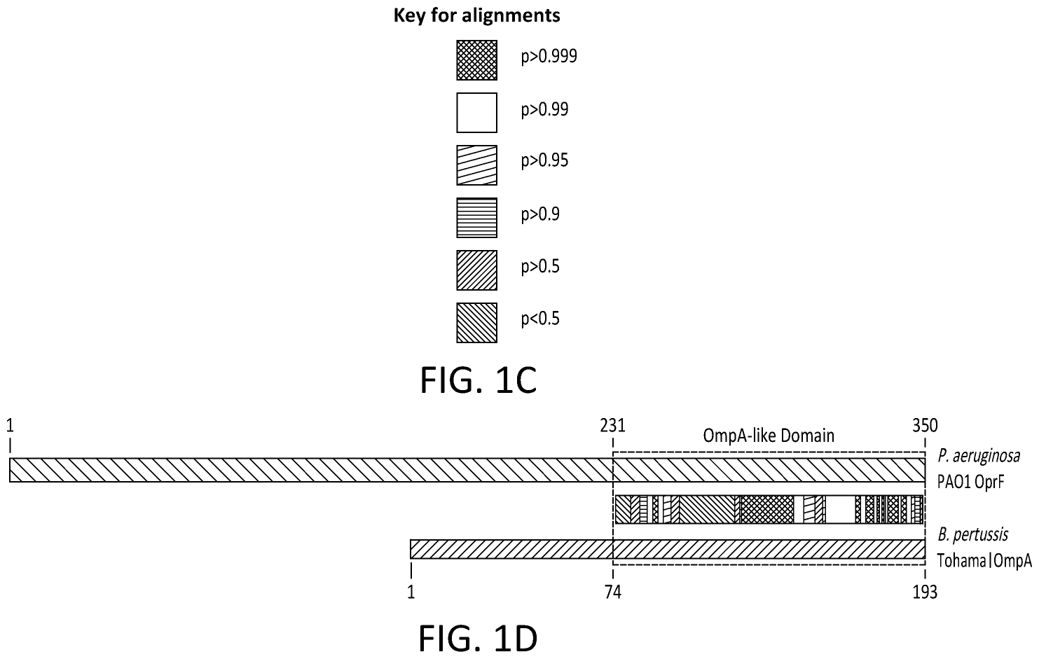

FIG. 1A shows the alignment between the sequences of B. pertussis GroEL (SEQ ID NO: 16) and P. aeruginosa GroEL (SEQ ID NO: 14), where the color-coded key for percentage identity along the length of the sequences is shown in FIG. 1C. FIG. 1B shows the alignment between the sequences of B. pertussis GroEL and E. coli GroEL (SEQ ID NO: 17), where the key for percentage identity is in FIG. 1C. As can be seen in FIGS. 1A and 1B, the amino acid sequences of B. pertussis GroEL and P. aeruginosa GroEL have 76% identity along their entire length. The sequences of B. pertussis GroEL and P. aeruginosa GroEL, and E. coli GroEL have greater than 95% identity along the majority of their length. Thus, an antibody to a GroEL chaperonin protein from any one of these three species is likely to have significant cross reactivity against GroEL proteins from all three of B. pertussis, P. aeruginosa, and E. coli. Additionally, a variety of drug resistant ESKAPE pathogens, including Enterococcus, Staphylococcus aureus, Klebsiella pneumonia, Acinetobacter baumanii, Enterobacter species, and Gammaproteobacter species, express GroEL homologs having 50% to 98%, 55% to 95%, 60% to 95%, 65% to 90%, 70% to 85%, 75% to 80%, 80% to 95%, 90% to 95%, or 92% to 95% identity to the sequence of GroEL as expressed by the PAO1 P. aeruginosa strain, as shown in Table 1. Thus, GroEL antigenic proteins from a single bacterial species may induce formation of antibodies which exhibit cross reaction against a range of bacterial pathogens.

In various embodiments, antibodies against fragments of B. pertussis GroEL having SEQ ID NO: 16 may be used to induce cross reactivity against P. aeruginosa bacteria expressing GroEL, where the antibodies may be generated against fragments of SEQ ID NO: 16 having from 10 to 520, from 25 to 500, from 45 to 450, from 75 to 400, from 100 to 350, from 150 to 300, from 175 to 275, or from 200 to 250 continuous residues of SEQ ID NO: 16.

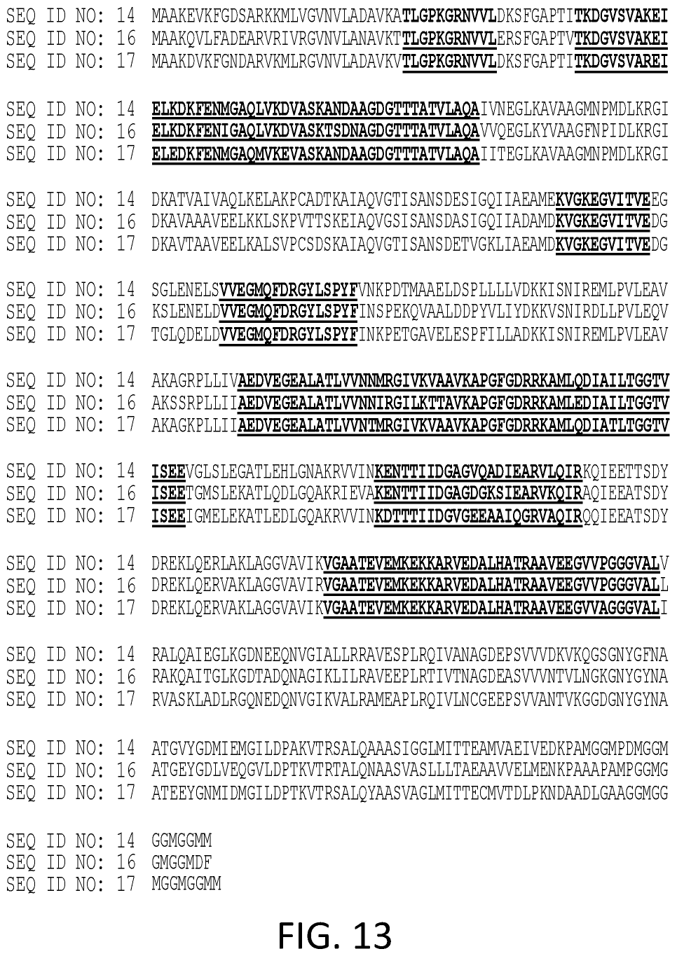

FIG. 13 shows alignment between GroEL of B. pertussis (SEQ ID NO: 16), P. aeruginosa (SEQ ID NO: 14), and E. coli (SEQ ID NO: 17), where conserved sequences of interest are highlighted in bold text. In various embodiments, antibodies against fragments of B. pertussis GroEL having SEQ ID NO: 16 may be used to induce cross reactivity against P. aeruginosa bacteria expressing GroEL, where the antibodies may be generated against:
A B. pertussis GroEL fragment having at least 85%, 90%, or 95% identity to the sequence having amino acid residues 30 to 40 of SEQ ID NO: 16;
A B. pertussis GroEL fragment having at least 85%, 90%, or 95% identity to the sequence having amino acid residues 50 to 98 of SEQ ID NO: 16;
A B. pertussis GroEL fragment having at least 85%, 90%, or 95% identity to the sequence having amino acid residues 168 to 178 of SEQ ID NO: 16;
A B. pertussis GroEL fragment having at least 85%, 90%, or 95% identity to the sequence having residues 189 to 204 of SEQ ID NO: 16;
A B. pertussis GroEL fragment having at least 85%, 90%, or 95% identity to the sequence having residues 251 to 304 of SEQ ID NO: 16;
A B. pertussis GroEL fragment having at least 85%, 90%, or 95% identity to the sequence having residues 326 to 349 of SEQ ID NO: 16; or
A B. pertussis GroEL fragment having at least 85%, 90%, or 95% identity to the sequence having residues 381 to 419 of SEQ ID NO: 16.

In various embodiments, antibodies against fragments of P. aeruginosa GroEL having SEQ ID NO: 14 may be used to induce cross reactivity against other species of bacteria expressing GroEL, where the antibodies may be generated against a P. aeruginosa GroEL fragment having at least 85%, 90%, or 95% identity to the sequence having amino acid residues 30 to 40 of SEQ ID NO: 14; at least 85%, 90%, or 95% identity to the sequence having amino acid residues 50 to 98 of SEQ ID NO: 14; at least 85%, 90%, or 95% identity to the sequence having amino acid residues 168 to 178 of SEQ ID NO: 14; at least 85%, 90%, or 95% identity to the sequence having residues 189 to 204 of SEQ ID NO: 14; at least 85%, 90%, or 95% identity to the sequence having residues 251 to 304 of SEQ ID NO: 14; at least 85%, 90%, or 95% identity to the sequence having residues 326 to 349 of SEQ ID NO: 14; or at least 85%, 90%, or 95% identity to the sequence having residues 381 to 419 of SEQ ID NO: 14.

In various embodiments, antibodies against fragments of E. coli GroEL having SEQ ID NO: 17 may be used to induce cross reactivity against other species of bacteria expressing GroEL, where the antibodies may be generated against a E. coli GroEL fragment having at least 85%, 90%, or 95% identity to the sequence having amino acid residues 30 to 40 of SEQ ID NO: 17; at least 85%, 90%, or 95% identity to the sequence having amino acid residues 50 to 98 of SEQ ID NO: 17; at least 85%, 90%, or 95% identity to the sequence having amino acid residues 168 to 178 of SEQ ID NO: 17; at least 85%, 90%, or 95% identity to the sequence having residues 189 to 204 of SEQ ID NO: 17; at least 85%, 90%, or 95% identity to the sequence having residues 251 to 304 of SEQ ID NO: 17; at least 85%, 90%, or 95% identity to the sequence having residues 326 to 349 of SEQ ID NO: 17; or at least 85%, 90%, or 95% identity to the sequence having residues 381 to 419 of SEQ ID NO: 17.

In various embodiments, antibodies against fragments of P. aeruginosa GroEL having amino acid residues 30 to 40, 50-98, 168 to 178, 189 to 204, 251 to 304, 326 to 349, or 381 to 419 of SEQ ID NO: 16 may be used to induce an immune response against:
an ESKAPE pathogen, such as a bacteria from a genus Pseudomonas, a genus Escherichia, a genus Enterococcus, a genus Staphylococcus, a genus Klebsiella, a genus Acinetobacter, and a genus Enterobacter;
Escherichia coli; and/or
a gram-negative bacteria.

In various embodiments, antibodies against fragments of B. pertussis GroEL having amino acid residues 30 to 40, 50-98, 168 to 178, 189 to 204, 251 to 304, 326 to 349, or 381 to 419 of SEQ ID NO: 16 may be used to induce an immune response against:
an ESKAPE pathogen, such as a bacteria from a genus Pseudomonas, a genus Escherichia, a genus Enterococcus, a genus Staphylococcus, a genus Klebsiella, a genus Acinetobacter, and a genus Enterobacter;
B. pertussis;
Escherichia coli; and/or
a gram-negative bacteria.

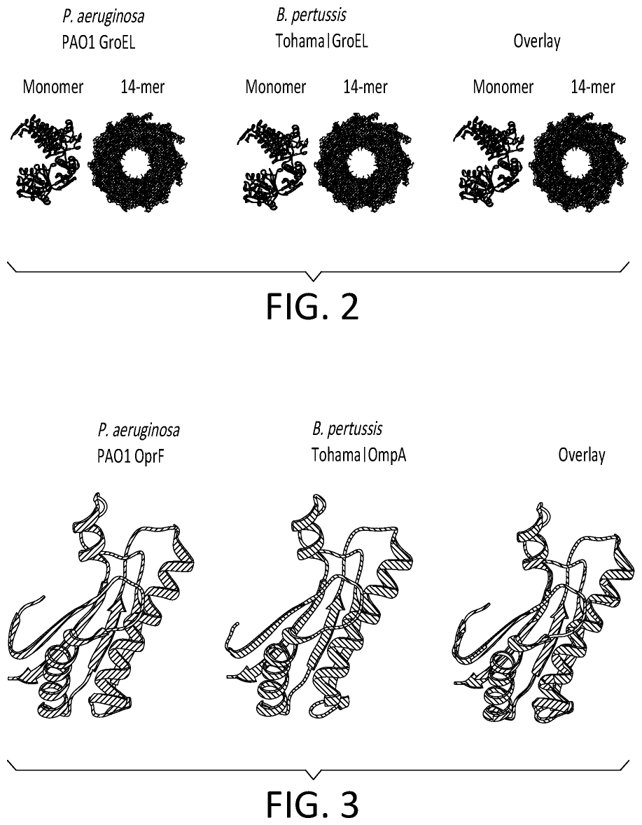

FIG. 2 shows:
the conformational structure of a monomeric P. aeruginosa GroEL protein having SEQ ID NO: 14, and the conformational structure of a P. aeruginosa GroEL protein having 14 monomeric peptide units of SEQ ID NO: 14 (a 14-mer);
the conformational structure of a monomeric B. pertussis GroEL protein having SEQ ID NO: 16, and the conformational structure of a B. pertussis GroEL 14-mer;
an overlay of the monomeric P. aeruginosa and B. pertussis GroEL protein; and
an overlay of the P. aeruginosa and B. pertussis GroEL 14-mers.

When the structure of these proteins was predicted using SWISS-MODEL homology modeling, both were modelled based on the crystallized structure of GroEL from Xanthomonas oryzae (PDB ID 6KFV), and showed high structural similarity, lending further support to the idea that an antibody exhibiting reactivity to either monomeric GroEL or a GroEL 14-mer from either of P. aeruginosa or B. pertussis would exhibit cross reactivity to a corresponding GroEL species from the other pathogen.

FIG. 1D shows the alignment between the sequence of B. pertussis Tohama I OmpA (SEQ ID NO: 8) and P. aeruginosa PAO1 OprF (SEQ ID NO: 2). The color-coded key for percentage identity along the length of the sequences is shown in FIG. 1C. Overall, there is 46% identity between:
SEQ ID NO: 3, the OmpA-like domain of the OprF protein of Pseudomonas aeruginosa including amino acid residues 231 to 350 of SEQ ID No: 2; and
SEQ ID NO: 9, a fragment of the OmpA protein of Bordetella pertussis including amino acid residues 74 to 193 of SEQ ID No: 8.

Thus, in various embodiments, antibodies against B. pertussis OmpA may exhibit cross reactivity against P. aeruginosa bacteria expressing OprF, and vice versa. Similarly, B. pertussis OmpA may serve as an antigen against a variety of bacteria expressing proteins with OmpA-like domains. Also, antibodies against the 120 residue-containing B. pertussis OmpA fragment having SEQ ID NO: 9 may exhibit cross reactivity against P. aeruginosa bacteria expressing OprF, while a Pseudomonas aeruginosa antigen having SEQ ID NO: 3 may exhibit cross reactivity against Bordetella pertussis bacteria.

As seen in FIG. 1D, there is generally at least 90% identity between:
a fragment of the OprF protein of Pseudomonas aeruginosa having SEQ ID NO: 6, including amino acid residues 278 to 344 of SEQ ID No: 2.
a fragment of the OmpA protein of Bordetella pertussis having SEQ ID NO: 12, including amino acid residues 121 to 186 of SEQ ID No: 8.

Thus, in various embodiments, antibodies against the 66 residue-containing B. pertussis OmpA fragment having SEQ ID NO: 12 may exhibit cross reactivity against P. aeruginosa bacteria expressing OprF, while a Pseudomonas aeruginosa antigen having the 67 residue-containing OprF fragment having SEQ ID NO: 6 may exhibit cross reactivity against Bordetella pertussis bacteria.

As seen in FIG. 1D, there is generally at least 99% identity between:
a fragment of the OprF protein of Pseudomonas aeruginosa having SEQ ID NO: 4, including amino acid residues 278 to 303 of SEQ ID No: 2.
a fragment of the OmpA protein of Bordetella pertussis having SEQ ID NO: 10, including amino acid residues 121 to 145 of SEQ ID No: 8.

Thus, in various embodiments, antibodies against the 25 residue-containing B. pertussis OmpA fragment having SEQ ID NO: 10 may exhibit cross reactivity against P. aeruginosa bacteria expressing OprF, while a Pseudomonas aeruginosa antigen having the 26 residue-containing OprF fragment having SEQ ID NO: 4 may exhibit cross reactivity against Bordetella pertussis bacteria.

As seen in FIG. 1D, there is generally at least 95% identity between:
a fragment of the OprF protein of Pseudomonas aeruginosa having SEQ ID NO: 5, including amino acid residues 328 to 344 of SEQ ID No: 2.
a fragment of the OmpA protein of Bordetella pertussis having SEQ ID NO: 11, including amino acid residues 170 to 186 of SEQ ID No: 8.

Thus, in various embodiments, antibodies against the 17 residue-containing B. pertussis OmpA fragment having SEQ ID NO: 11 may exhibit cross reactivity against P. aeruginosa bacteria expressing OprF, while a Pseudomonas aeruginosa antigen having the 17 residue-containing OprF fragment having SEQ ID NO: 5 may exhibit cross reactivity against Bordetella pertussis bacteria.

In various embodiments, antibodies against fragments of B. pertussis OmpA having SEQ ID NO: 9 may be used to induce cross reactivity against P. aeruginosa bacteria expressing OprF, where the antibodies may be generated against fragments of SEQ ID NO: 9 having from 10 to 110, from 17 to 100, from 20 to 90, from 25 to 80, from 30 to 70, from 40 to 65, or from 45 to 60 continuous residues of SEQ ID NO: 9.

As noted above, alignment of the OmpA domain of the P. aeruginosa OprF protein and the B. pertussis OmpA protein showed that there was a 46% identity between the amino acid sequences of the two OmpA domains (FIG. 1D). To further understand the similarity of these proteins, the structures of the P. aeruginosa OmpA domain and B. pertussis OmpA were compared. The structure of the OmpA domain of P. aeruginosa OprF was previously solved using x-ray crystallography (PDB ID 5U1H), and is shown in FIG. 3. The SWISS-MODEL homology modeling system was used to predict the structure of B. pertussis OmpA, and the predicted structure, as visualized using UCSF Chimera software, is shown in FIG. 3. Overlay of the structures showed a high structural homology between B. pertussis OmpA and the OmpA domain of P. aeruginosa OprF (FIG. 3), lending further support to the idea that an antibody exhibiting reactivity to either B. pertussis OmpA or the OmpA domain of P. aeruginosa would exhibit cross reactivity to a corresponding species from the other pathogen.

In various embodiments, the full B. pertussis OmpA protein having SEQ ID NO: 8 may be used as an antigen to generate cross reactive antibodies exhibiting activity against pathogens expressing an OprF protein with an OmpA-like domain, e.g., ESKAPE pathogens in general, and Pseudomonas aeruginosa in particular. Similarly, a B. pertussis OmpA protein fragment having SEQ ID NO: 9, 10, 11, or 12 may be used to generate cross reactive antibodies exhibiting activity against pathogens expressing an OprF protein with an OmpA-like domain. The B. pertussis OmpA protein, or a suitable fragment thereof, may be administered intranasally, intravenously, intramuscularly, subcutaneously, intradermally, orally, rectally, or intraperitoneally in a suitable vehicle, e.g., an aqueous vehicle such as water or saline. The B. pertussis OmpA protein, or a suitable fragment thereof, may be formulated with a suitable adjuvant, e.g., curdlan, alum, amorphous aluminum hydroxyphosphate sulfate, aluminum hydroxide, aluminum phosphate, monophosphoryl lipid A, squalene, cytosine phosphoguanine, or combinations thereof.

In various embodiments, the full OprF protein of Pseudomonas aeruginosa having SEQ ID NO: 2 may be used as an antigen to generate cross reactive antibodies exhibiting activity against other species of pathogens expressing OmpA or a protein with an OmpA-like domain, e.g., ESKAPE pathogens and Bordetella pertussis. Similarly, a P. aeruginosa OprF protein fragment having SEQ ID NO: 3, 4, 5, or 6 may be used to generate cross reactive antibodies exhibiting activity against ESKAPE pathogens and Bordetella pertussis. The P. aeruginosa OprF protein, or a suitable fragment thereof, may be administered intramuscularly, intraperitoneally, or intranasally in a suitable vehicle, e.g., an aqueous vehicle such as water or saline. The P. aeruginosa OprF protein, or a suitable fragment thereof, may be formulated with a suitable adjuvant, e.g., curdlan, alum, amorphous aluminum hydroxyphosphate sulfate, aluminum hydroxide, aluminum phosphate, monophosphoryl lipid A, squalene, cytosine phosphoguanine, SWE, or combinations thereof.

In a similar manner, the GroEL protein of one species, e.g., Pseudomonas aeruginosa, Escherichia coli, or Bordetella pertussis, may be used as an antigen to generate cross reactive antibodies exhibiting activity against other species of pathogens expressing a GroEL protein. The antigenic GroEL protein, or a suitable fragment thereof, may be administered intramuscularly, intraperitoneally, or intranasally in a suitable vehicle, e.g., an aqueous vehicle such as water or saline. The GroEL protein, or a suitable fragment thereof, may be formulated with a suitable adjuvant, e.g., curdlan, alum, amorphous aluminum hydroxyphosphate sulfate, aluminum hydroxide, aluminum phosphate, monophosphoryl lipid A, squalene, cytosine phosphoguanine, SWE, or combinations thereof.

Antigenic GroEL proteins, OprF, proteins, OmpA proteins, or fragments thereof, may be used as unmodified peptide sequences to generate an immune response. Alternatively, such antigenic peptides may be used as a hapten, and modified by conjugation to a carrier protein through a linking group. A suitable carrier protein may be CRM197 (genetically detoxified form diphtheria toxin), rTTHc (Tetanus toxoid heavy chain fragment C), KLH (Keyhole limpet hemocyanin), thyroglobulin from bovine thyroid, or a combination thereof.

In various embodiments, an antigenic protein selected from:
B. pertussis GroEL,
B. pertussis OmpA,
P. aeruginosa OprF,
P. aeruginosa GroEL,
GroEL proteins from other bacterial pathogens,
proteins having OmpA domains from other bacterial pathogens,
fragments thereof, or mixtures thereof
may be modified with an amino acid tag to allow for improved protein purification, metal chelation, or binding assays. Suitable amino acid tags may be bound to the protein N-terminus, the protein C-terminus, or both. Suitable amino acid tags include a polyhistidine tag, e.g., HHHHHH; an HQ tag, e.g., HQHQHQ; an HN tag, e.g., HNHNHNHNHNHN; a glutathione S transferase tag; a maltose binding protein tag; and an HAT tag derived from chicken lactate dehydrogenase, e.g., KDHLIHNVHKEEHAHAHNK.

The antigenic hapten may be conjugated to the carrier protein through a linking group. Suitable linking groups include amino acid chains having from 1 to 10 residues, including a cysteine or other residue including a sulfhydryl group. The sulfhydryl group on the hapten is then reacted with a carrier protein with a reactive maleimide moiety. Alternative linking groups may include amino acid or alkylene chains with amino or carboxyl groups. A carbodiimide reagent is used to link the amino or carboxyl group on the linking group with a corresponding reactive group on the desired carrier protein. For example, the carbodiimide may be used to form an amide bond between an amino group on the hapten and a carboxyl group on the carrier protein.

In various embodiments, antigenic GroEL proteins, OprF, proteins, OmpA proteins, or fragments thereof, may be produced by expression in E. coli as fusion proteins with maltose binding protein, using methods known in the art.

In various embodiments, an antigenic protein selected from B. pertussis GroEL, B. pertussis OmpA, or mixtures thereof may be prepared by purifying GroEL and/or OmpA from a B. pertussis cell culture, and used as antigens in preparation of a vaccine against a bacterial pathogen expressing:
a peptide having at least 45%, 50%, 55%, 60%, 70%, 75%, 80%, 85%, or 90% identity to B. pertussis GroEL; and/or
a peptide having an OmpA domain with at least 45%, 50%, 55%, 60%, 70%, 75%, 80%, 85%, or 90% identity to SEQ ID NO: 9 from B. pertussis OmpA.

In various embodiments, an antigenic protein selected from P. aeruginosa GroEL, P. aeruginosa OmpA, or mixtures thereof may be prepared by purifying GroEL and/or OmpA from a P. aeruginosa cell culture, and used as antigens in preparation of a vaccine against a bacterial pathogen expressing:
a peptide having at least 45%, 50%, 55%, 60%, 70%, 75%, 80%, 85%, or 90% identity to P. aeruginosa GroEL; and/or
a peptide having an OmpA domain with at least 45%, 50%, 55%, 60%, 70%, 75%, 80%, 85%, or 90% identity to SEQ ID NO: 3 from P. aeruginosa OprF.

In various embodiments, DNA having SEQ ID NO: 16, which encodes the GroEL protein of Bordetella pertussis, may be inserted into viral DNA under the control of a promoter, and the viral DNA may be incorporated into a vector, e.g., an adenoviral vector. The vector with the DNA encoding Bordetella pertussis GroEL may be administered to a patient to induce production of GroEL antigens to induce an immune response against Pseudomonas aeruginosa or other ESKAPE pathogens. Similarly, DNA having SEQ ID NO: 7, which encodes the OmpA protein of Bordetella pertussis, may be inserted into viral DNA under the control of a promoter, and used to generate an immune response against Pseudomonas aeruginosa or other ESKAPE pathogens expressing proteins with OmpA-like domains. Fragments of DNA having SEQ ID NO: 16 or SEQ ID NO: 7 encoding desired antigenic peptides may be used in place of the complete sequences.

In a similar fashion, SEQ ID NO: 1, the DNA sequence encoding the OprF protein of Pseudomonas aeruginosa, SEQ ID NO: 13, the DNA sequence encoding the GroEL protein of Pseudomonas aeruginosa, and/or fragments thereof, may be inserted into viral DNA under the control of a promoter, and used to generate an immune response against Bordetella pertussis. Fragments of DNA having SEQ ID NO: 1 or SEQ ID NO: 13 encoding desired antigenic peptides may be used in place of the complete sequences.

Taken altogether, this disclosure shows that cross-reactivity amongst antigens between antigenic proteins in vaccines against a first pathogen, e.g., B. pertussis, may be useful in creating or enhancing an immune response against an unrelated pathogens expressing a structurally similar peptide.

### EXAMPLES

In the following examples, the P. aeruginosa strain PAO1 was used for vaccine preparation and murine challenges. For assays using transposon mutants, the University of Washington Transposon Library of PAO1 transposon insertion mutants was utilized; the transposon mutants used in this work are presented in Table 2. To prepare a challenge dose of P. aeruginosa, PAO1 was grown on the Miller formulation of lysogeny agar (LA-Miller) overnight at 36° C. A single colony was used to start 3 mL cultures, which were incubated while shaking, overnight at 36° C. To prepare a culture in an exponential phase, aliquots of the overnight culture were diluted 1:100 in fresh lysogeny broth (LB), allowed to grow for approximately 4 to 5 hours, then centrifuged, resuspended in sterile 1× phosphate buffered saline (PBS), and diluted to an infectious dose of 3-5×10⁷ CFU/20 μL, using optical density (OD₆₀₀). The dose was quantified using serial dilution and plating on Pseudomonas isolation agar (PIA).

TABLE 2
Transposon Mutant Strains of P. aeruginosa used in study.

| Gene ID | Strain | Pa ORF | Position in ORF | Transposon |
|---|---|---|---|---|
| oprF-1 | PW4134 | PA1777 | 432(1053) | ISlacz/hah |
| oprF-2 | PW4135 | PA1777 | 124(1053) | ISlacZ/hah |

PAO1 transposon mutant and parental strains were struck on LA-Miller supplemented with tetracycline (50 μg/mL). Clinical isolates of P. aeruginosa, described by Burns et al., were kindly provided by Dr. Robert Ernst (Table 3). Strains were grown on PIA overnight at 37° C.

TABLE 3
Clinical isolate strains used, and their phenotypes, described by Burns et al.

| Strain | | | Colony | Colony | | | |
|---|---|---|---|---|---|---|---|
| ID | Source | Mucoid | Shape | Size | Color | Swimming | Rhamnolipid |
| CEC34 | BAL | M | Irregular | Large | Mint | >1 cm | + |
| CEC38 | OP | NM | Round | Small | Cucumber | >1 cm | + |
| CEC45 | OP | NM | Irregular | Small | Cucumber | Surface | + |
| | | | | | | swimming | |
| CEC55 | OP | M | Round | Large | Cucumber | <1 cm | + |
| CEC65 | OP | M | Round | Large | Sea Spray | <1 cm | +/− |
| CEC76 | OP | M | Round | Medium | Cucumber | >1 cm | + |
| CEC86 | BAL | NM | Irregular | Small | Cucumber | <1 cm | + |

BAL—bronchioalveolar lavage,
OP—oropharyngeal swab,
M—mucoid,
NM—non-mucoid

Proteins of interest were visualized in UCSF Chimera software. Protein structures for P. aeruginosa OprF (PDB ID: 4RLC) and the OmpA domain of P. aeruginosa OprF (PDB ID: 5U1H; the C-terminal peptidoglycan binding domain of OprF from Pseudomonas aeruginosa) were downloaded from the Protein Data Bank (PDB). Homology modelling of B. pertussis OmpA was performed using SWISS-MODEL homology modeling server, and the highest homology protein was selected for reference (PDB ID: 5U1H). Both GroEL structures depicted were modelled similarly using SWISS-MODEL, and based on GroEL from Xanthomonas oryzae (PDB ID: 6KFV). In comparing P. aeruginosa and X. oryzae protein sequences, there was a 78.05% sequence identity, and the model had a QMQE score of 0.80 and QMEAN of −0.15. Similarly, the B. pertussis GroEL had 75.19% identity with the X. oryzae protein sequence, and the model had a QMQE score of 0.79 and QMEAN of −0.67. Structures were visualized and overlaid using the MatchMaker structure analysis tool in UCSF Chimera software.

### Example 1. Whole Cell Vaccine Preparation

Pseudomonas aeruginosa whole cell vaccine (Pa-WCV) and Bordetella pertussis whole cell vaccine (Bp-WCV) were prepared. P. aeruginosa PAO1 Vasil was grown on Pseudomonas isolation agar (PIA) overnight, swabbed from the Petri dish into sterile, endotoxin-free phosphate buffered saline (PBS), diluted, and heat-killed by incubation for 1 hour at 60° C. Vaccines were formulated to include 20 μL of the killed bacterial suspension.

For vaccination against B. pertussis, the National Institute for Biological Standards and Control (NIBSC) whole cell vaccine was used, and Diphtheria and Tetanus Toxoids and Acellular Pertussis Vaccine Adsorbed (Infanrix®) suspension was used as an acellular vaccine (Bp-ACV), each diluted to 1/50th of the human dose.

Vaccines were formulated with either 200 μg of the adjuvant curdlan or 62.5 μg of the adjuvant alum, and used in a total volume of 40 μL if administered intranasally, 50 μL if administered intramuscularly or intraperitoneally. Curdlan was prepared by methods known in the art, and mixed into the vaccine immediately before administration. Alum adjuvanted vaccines were allowed to adsorb for 1 hour at room temperature, with rotation, and were thoroughly mixed immediately prior to administration to animals.

### Example 2. Murine Immunization and Challenge Models

To examine efficacy of the vaccines in an out-bred mouse model, groups of 6-week-old CD-1 (Charles River) female mice were used. To examine efficacy in a more clinically relevant model for cystic fibrosis, groups of transgenic β-ENaC mice were used, in comparison to wild-type (WT) C57BL/6 littermates. For these studies, 13- to 15-week-old female mice, age matched per group, were used.

Pa-WCV, Bp-WCV, and Infanrix® acellular vaccines were administered either intranasally under anesthesia or intraperitoneally, and compared to adjuvant-only vaccination. Vaccines were administered to mice on days 0 and 21. Thirty-four days post initial vaccination, mice were anesthetized, and infected intranasally with 20 μL of bacterial culture containing 3-5×107 CFU PAO1. Approximately 14-16 hours post-infection, mice were euthanized by IP injection of 390 mg pentobarbital/kg (Patterson Veterinary) in 0.9% NaCl. Following euthanasia, blood was collected via cardiac puncture. Lung and spleen were aseptically removed and weighed prior to any further treatment or analysis. Nasal lavage was collected by pushing 1 mL of sterile PBS through the nasal cavity.

Bacteria were quantified in the lung and nasal wash. The nasal wash was serially diluted and plated on PIA, then grown overnight at 36° C. The lung was homogenized using a polytron PT 2500 E homogenizer, then similarly serially diluted, plated, and grown overnight.

A. B. pertussis Whole Cell Vaccine Protects Outbred CD-1 Mice from Acute P. aeruginosa Respiratory Infection.

Mice were vaccinated and boosted on days 0 and 21, and infected intranasally with P. aeruginosa at day 35. Mice were vaccinated with:
curdlan alone,
Pa-WCV+curdlan,
Bp-WCV+curdlan, and
Pa-WCV+Bp-WCV+curdlan.

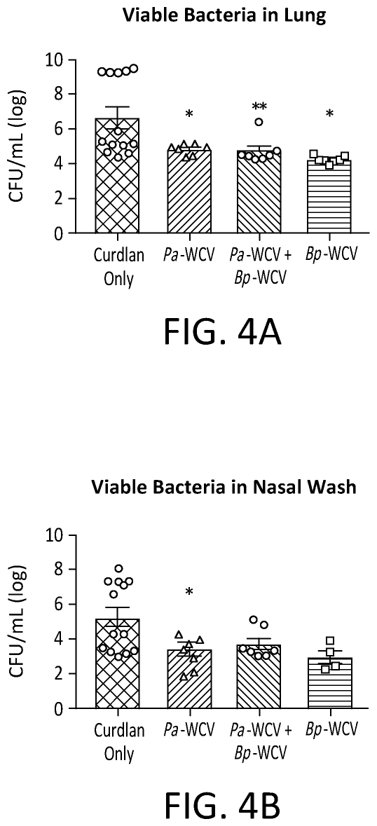

One day post-infection, animals were euthanized and bacterial burden in the respiratory tract was determined. Curdlan-only vaccinated mice were heavily colonized in both the lung and nasal wash (FIGS. 4A and 4B). In comparison to curdlan-only vaccinated, both the Pa-WCV+curdlan and the combined Pa-WCV+Bp-WCV+curdlan vaccinated animals had a significant reduction of bacterial burden in the lung (FIG. 4A). However, combination of both vaccines did not provide additional protection compared to Pa-WCV alone. Surprisingly, the animals which had received only the Bp-WCV also showed a significant reduction in P. aeruginosa bacterial burden in the lung, indicating that vaccination with Bp-WCV alone is sufficient for protection against P. aeruginosa (FIG. 4A). Similar results were observed by nasal lavage (FIG. 4B).

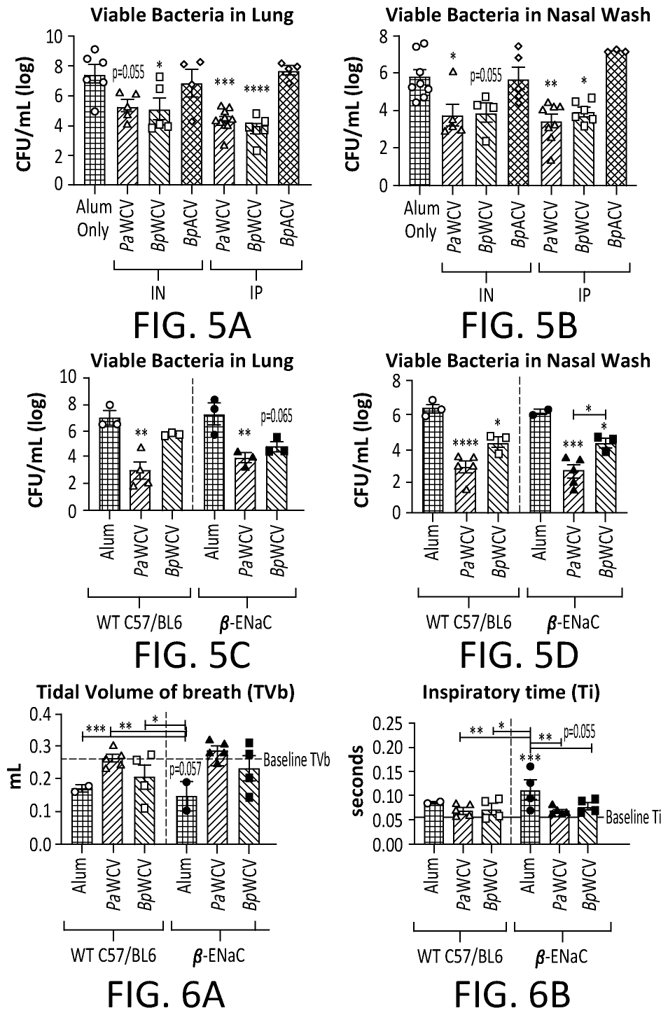

Bp-WCV is classically formulated with the adjuvant alum and administered intramuscularly in the human population. To determine if Bp-WCV provides cross-protection in a model more closely related to what is currently used in humans, and determine if protection is adjuvant- or route-dependent, both Pa-WCV and Bp-WCV were reformulated with alum, and administered both intranasally and intraperitoneally. As a control, the efficacy of acellular pertussis vaccine (Bp-ACV) administration using intranasal and intraperitoneal routes was tested. Animals which had received alum-only vaccination were heavily colonized in their upper and lower respiratory tracts, as determined by quantification of colony forming units (CFU) in the nasal wash and lung homogenate (FIGS. 2A and 2B). Regardless of the route of administration, the Pa-WCV led to a significant reduction of bacterial burden in both the lung and nasal wash (FIGS. 5A and 5B). The groups which received Bp-WCV alone had a significant reduction of P. aeruginosa bacterial burden in both the lung and nasal wash (FIGS. 5A and 5B), indicating that the Bp-WCV induced cross-protection against P. aeruginosa when the Bp-WCV vaccine was administered with the adjuvant alum. This cross-protection was not observed with the acellular pertussis vaccine (FIGS. 5A and 5B). Overall, Bp-WCV was protective against P. aeruginosa regardless of the route of administration or the adjuvant formulation.

B. B. pertussis Whole Cell Immunization Induces Protection in a β-ENaC Murine Model.

P. aeruginosa is known for its ability to cause difficult-to-treat antibiotic resistant infections in the lung of patients with cystic fibrosis (CF). Vaccination against P. aeruginosa could be particularly beneficial in these patients to reduce the burden of disease and improve morbidity and mortality associated with P. aeruginosa infections. To determine if the observations in outbred CD-1 mice also translate to a CF-like model, mice overexpressing the β-ENaC receptor, which displays CF-like lung phenotypes were compared to their wild type C57BL/6 littermates. Mice were vaccinated with Pa-WCV, Bp-WCV, or the adjuvant alum intraperitoneally on days 0 and 21, and then challenged with P. aeruginosa intranasally. Pa-WCV was able to induce a protective immune response against acute P. aeruginosa infection in both C57BL/6 and β-ENaC mice 14 hours post-infection (FIGS. 5C and 5D). In addition, Bp-WCV administration also significantly reduced P. aeruginosa bacterial burden in the nasal wash in both strains (FIG. 5D).

### Example 3. Serology

Antibody titers were quantified using an enzyme-linked immunosorbent assay (ELISA). 96-well microtiter plates were coated with 500_, of PBS containing 2×10⁷ CFU grown overnight on PIA. Following coating, plates were washed thrice with PBS+0.05% Tween 20 (Fisher Scientific) (PBS-T), then blocked using 2% weight/volume (w/v) bovine serum albumin (BSA) overnight at 4° C. Serum samples were prepared by diluting in 2% w/v BSA at a dilution of 1:50, diluted serially down the plate to a maximum dilution of 1:819200, and incubated overnight at 4° C. Following treatment with the serum, plates were washed four times with PBS-T. Anti-IgG secondary antibody conjugated to alkaline phosphatase (SouthernBiotech) was diluted 1:2,000 in blocking buffer and 100 μL were added to each well, then incubated at 36° C. for one hour. Plates were washed 5 times with PBS-T, then incubated with Pierce p-Nitrophenyl Phosphate (PNPP) (Thermo Fisher Scientific) for 30 minutes, per the manufacturer's instructions. Optical density at 405 nm (OD405) was quantified using SpectraMax i3 (Molecular Devices LLC). Titer was quantified by calculating the highest dilution at which the OD405 signal was double that of the blank, and any samples for which no signal was detected was assigned a value of 1.

A. B. pertussis Whole Cell Immunization Induces the Production of Anti-P. aeruginosa Antibodies.

In the case of P. aeruginosa, antibodies induced by Pa-WCV are a mechanistic correlate of protection. Vaccination with Bp-WCV generates antibodies that bind the surface of P. aeruginosa. To characterize the antibody response induced by these vaccine formulations, an ELISA assay was used to detect serum IgG binding to whole P. aeruginosa bacteria.

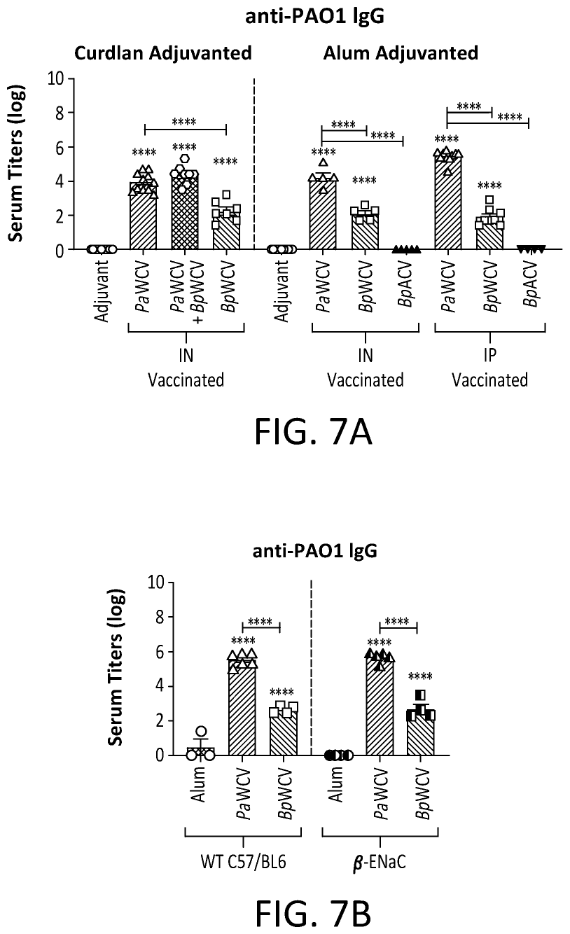

CD-1 mice that received the adjuvant alone, curdlan or alum, regardless of whether intranasal or intraperitoneal route of administration, did not produce detectable anti-P. aeruginosa antibodies (FIG. 7A). However, vaccination of CD-1 mice using Pa-WCV vaccine induced significant levels of anti-P. aeruginosa IgG in the serum. Similar results were seen with:
Intranasal vaccination with Pa-WCV using a curdlan adjuvant;
Intranasal vaccination with Pa-WCV using an alum adjuvant; and
Intraperitoneal vaccination with Pa-WCV using an alum adjuvant.

In addition, the Bp-WCV alone induced production of cross-reactive anti-P. aeruginosa IgG in all these models, supporting our hypothesis (FIG. 7A). Vaccination of CD-1 mice using Bp-WCV vaccine induced significant levels of anti-P. aeruginosa IgG in the serum. Similar results were seen with:
Intranasal vaccination with Bp-WCV using a curdlan adjuvant;
Intranasal vaccination with Bp-WCV using an alum adjuvant; and
Intraperitoneal vaccination with Bp-WCV using an alum adjuvant.

In contrast, Bp-ACV vaccination with an alum adjuvant did not lead to the production of detectable anti-P. aeruginosa antibodies, regardless of whether the acellular vaccine was administered intranasally or intraperitoneally (FIG. 7A).

Intranasal vaccination using a combination of Pa-WCV and Bp-WCV with a curdlan adjuvant led to the production of anti-P. aeruginosa antibodies; however, the use of Pa-WCV and Bp-WCV did not provide a significantly greater effect than Pa-WCV alone (FIG. 7A).

To determine if the observations in outbred CD-1 mice also translate to a CF-like model, mice overexpressing the β-ENaC receptor were compared to their wild type C57BL/6 littermates. Vaccination of C57BL/6 mice using either Bp-WCV or Pa-WCV vaccines induced significant levels of anti-P. aeruginosa IgG in the serum, while vaccination with the alum adjuvant alone did not produce detectable anti-P. aeruginosa antibodies. FIG. 7B presents results obtained using:
Intraperitoneal vaccination with Pa-WCV using an alum adjuvant; and
Intraperitoneal vaccination with Bp-WCV using an alum adjuvant.

In addition, both the Bp-WCV or Pa-WCV vaccines induced production of cross-reactive anti-P. aeruginosa IgG in mice overexpressing the β-ENaC receptor, while vaccination with alum alone produced no detectable IgG. FIG. 7B presents results obtained using:
Intraperitoneal vaccination with Pa-WCV using an alum adjuvant; and
Intraperitoneal vaccination with Bp-WCV using an alum adjuvant.

### Example 4. Whole Body Plethysmography

To examine the impact of infection, with or without prior vaccination, on the breathing and respiratory function of animals, WT and β-ENaC transgenic mice were studied in a Buxco Small Animal Whole Body Plethysmography (WBP) chamber (Data Sciences International). Animals were acclimated to the chamber during a 20-minute session in the week prior to the recorded WBP sessions. A baseline measurement of breathing was recorded the day prior to infection, and then post-infection breathing was monitored immediately prior to euthanasia, 15 hours post-infection. All WBP sessions included 5 minutes of acclimation, then data were recorded for 15 minutes. Data was compiled and analyzed using FinePointe software, then exported to GraphPad Prism for statistical analysis. Collected data include breaths per minute, enhanced pause, pause, EF50, tidal volume of breath (TVb), time of inspiration (Ti), and time of expiration (Te). Linear regression analysis was performed to identify which datasets correlated with bacterial burden.

To characterize the impact of the protective response induced by Pa-WCV, Bp-WCV, and the adjuvant alum on respiratory function, whole-body plethysmography (WBP) was used to calculate the tidal volume of the breath and the inspiration time in mice. This analysis revealed that infection significantly reduced the tidal volume of breath (TVb) in non-vaccinated mice regardless of the strain. We observed that vaccination with either Pa-WCV or Bp-WCV helped restore baseline TVb (FIG. 6A). Furthermore, the inspiration time (Ti), or time spent on inhaling, was significantly increased in infected β-ENaC transgenic mice, but not C57BL/6 mice, compared to the non-challenged baseline (FIG. 6B). Vaccination with either the Pa-WCV or Bp-WCV was able to resolve this increase and reduce Ti inspiration times to near non-challenged baseline values. Taken together, TVb and Ti data illustrate that P. aeruginosa infection led to longer, more shallow breathing in β-ENaC mice, and that both Pa-WCV and Bp-WCV administration restore TVb and Ti values in this phenotype to near baseline.

### Example 5. WCV-Vaccinated Serum Immunoblotting

To further examine the binding of antibodies in the polyclonal serum, we performed western blot analyses using serum from vaccinated or non-vaccinated groups against bacterial lysates and purified proteins. Bacteria of interest were grown as described above, swabbed into sterile PBS, lysed using sonication, and the protein concentration quantified using bicinchoninic acid assay (BCA) (Thermo Fisher). Unless otherwise designated, 10 μg of protein were added to Laemmli buffer (Bio-Rad)+355 mM β-mercaptoethanol, and boiled at 95° C. for 5 minutes. Samples were loaded into Invitrogen Novex WedgeWell 10% Tris-Glycine protein gel, and resolved by gel electrophoresis. Proteins were then transferred using a wet-transfer method onto rehydrated PVDF membranes (Bio-Rad), and the membranes were blocked overnight in 5% w/v skim milk in PBS-T at 4° C. Next, membranes were treated with pooled murine serum samples, at a concentration of 1:5000 in 1% w/v skim milk in PBS, for two hours at room temperature, with orbital shaking. The membranes were washed 3 times with PBS-T then treated with anti-IgG secondary antibody conjugated to horseradish peroxidase (HRP) (Immunoreagents) at a 1:5,000 concentration in 1% w/v skim milk in PBS, for 1 hour at room temperature with orbital shaking. The membranes were washed again 4 times in PBS-T and developed using SuperSignal West Femto Maximum Sensitivity Substrate (Thermo Scientific). Chemiluminescence signal was detected using a Chemidoc Touch Imaging System (Bio-Rad). For analysis, the chemiluminescence images were overlayed onto colorimetric images of the ladder, visualized, and analyzed using Image Lab software (Bio-Rad).

A. Immunoblot Serum IgG Antibody Binding to P. aeruginosa Lysate.

P. aeruginosa lysate was separated by SDS-PAGE and transferred to PVDF membrane. Membranes were blocked, then separated for primary incubation with pooled serum samples from Bp-WCV-, Pa-WCV-, and Bp-ACV vaccinated groups, indicated beneath the blot image of FIG. 8. Serum binding was detected using anti-IgG antibodies, developed, and imaged using Chemidoc Touch Imaging System (Bio-Rad). Chemiluminescent images were overlayed onto colorimetric images of ladder.

As seen in FIG. 8, IgG antibodies bind peptides from Pa-WCV-vaccinated serum, where the bound peptides include multiple peptides having molecular weights ranging from ˜30 kDal to ˜100 kDal. However, serum IgG antibodies bind only two major peptides from Bp-WCV-vaccinated serum, where the bound peptides include peptides having molecular weights of ˜30 kDal and ˜60 kDal. No serum IgG antibodies binding peptides from Bp-ACV-vaccinated serum are observed.

### Example 6. Immunoprecipitation and Mass Spectrometry

P. aeruginosa PAO1 was grown as described above, and 750 μg of PAO1 culture lysate was incubated overnight with pooled Bp-WCV serum. Immunoprecipitation was performed using the Pierce MS-Compatible Magnetic IP Kit and Protein A/G beads, per the manufacturer's instructions (Thermo Scientific).

To identify the P. aeruginosa antigens bound by antibodies triggered by Bp-WCV, which have molecular weights of ˜30 kDal and ˜60 kDal, immunoprecipitation and mass spectrometry were performed. Immunoprecipitants were formed by incubating Bp-ACV-vaccinated serum with whole P. aeruginosa lysate, and purifying antigen-bound antibodies using mass-spectrometry compatible magnetic protein A/G beads. The resulting purified immunoprecipitation products were analyzed by Liquid chromatography-mass spectrometry in the WVU Mass Spectrometry CORE Facility. Data were acquired on a Thermo Q Extractive mass-spectrometer, and peptides identified were mapped using Proteome Discoverer software version 2.3.0.523, using the SeQuestHT algorithm. The database used for mapping was Pseudomonas aeruginosa PAO1 SwissProt TaxID=208964. Only peptides of high confidence designation by SeQuestHT were considered.

This analysis led to the detection of 10 proteins with 2 or more peptides. The proteins were then sorted based on abundance and percent coverage of the antigen, and the top two outer-membrane protein candidates were selected for further analysis. The cross-reactive antigen candidates selected for further testing were:
P. aeruginosa GroEL with SEQ ID NO: 13 (25 peptides, 48% coverage); and
P. aeruginosa OprF with SEQ ID NO: 1 (7 peptides, 30% coverage).

GroEL is a chaperonin highly conserved across Gram-negative bacteria including Escherichia coli (80% identity) and antibiotic resistant bacteria such as the ESKAPE pathogens, as shown in Table 1 above.

P. aeruginosa outer membrane protein OprF is a protein of 37 kDal with two domains, an N-terminal 8-stranded beta-barrel domain which spans the outer membrane (20 kDal), and a C-terminal globular OmpA domain (15 kDal subunits, 56 kDal total). A pBLAST search of the whole OprF amino acid sequence within the Bordetella taxonomic identifier resulted in alignment of the OmpA domain of the P. aeruginosa OprF protein to the B. pertussis OmpA protein (BP0943).

### Example 7. P. aeruginosa Antigen Immunoblotting

A. Immunoblotting of Bp-WCV Serum to P. aeruginosa Lysate and Recombinant GroEL.

To identify the ˜60 kDal antigen observed via immunoblotting (FIG. 8), the molecular weight of the band observed to the proteins identified by immunoprecipitation and mass spectrometry. P. aeruginosa GroEL (SEQ ID NO: 13) had the highest number of peptides mapped during the mass spectrometry analysis (25 peptides, 48% coverage) and a similar molecular weight (57 kDal).

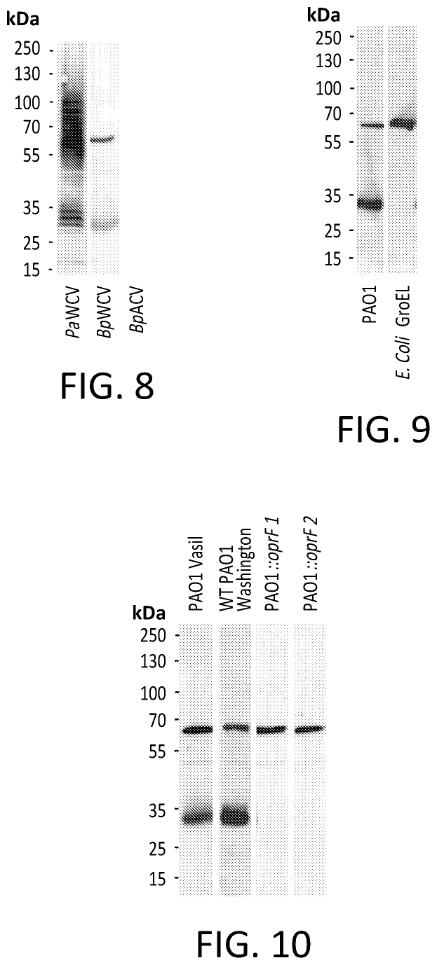

In the absence of readily accessible purified P. aeruginosa GroEL, immunoblotting using the closely related E. coli GroEL to determine whether Bp-WCV-induced antibodies could bind GroEL. Bp-WCV induced antibodies were capable of binding purified recombinant E. coli GroEL, as shown in FIG. 9. The observed band appeared to have a size of ˜60 kDal, similar to the PAO1 antigen observed via immunoblotting. Thus, the ˜60 kDal antigen observed via immunoblotting of Bp-WCV serum to P. aeruginosa lysate has been assigned to binding of P. aeruginosa GroEL.

B. Immunoblotting of Bp-WCV Serum to P. aeruginosa Lysate and OprF Mutants.

To identify if Bp-WCV-induced antibodies bind to either of these gene products, we performed immunoblotting on:
two oprF transposon mutants from the ordered PAO1 transposon mutant library (obtained from the Manoil Lab P. aeruginosa PAO1 transposon mutant library at the University of Washington),
the parental PAO1 strain (Washington, obtained from the Manoil Lab), and
a closely related PAO1 strain (Vasil, obtained from Dr. Mike Vasil, University of Colorado).

We observed that serum from Bp-WCV vaccinated mice reacted with a protein of approximately 60 kDal in the transposon mutants and the parental PAO1 strain (FIG. 10), which is assigned to binding to GroEL. In addition, the serum from Bp-WCV vaccinated mice also reacted with a protein of approximately 30 kDal in parental PAO1, but the binding was not detected against the PAO1::oprF1 and PAO1::oprF2 transposon mutant strains containing an insertion in the gene encoding OprF (FIG. 10).

Thus, the ˜30 kDal antigen observed via immunoblotting of Bp-WCV serum to P. aeruginosa lysate has been assigned to binding of the P. aeruginosa OprF protein.

### Example 8. Bp-WCV Induced Serum Antibodies Bind Clinical Isolates of P. aeruginosa

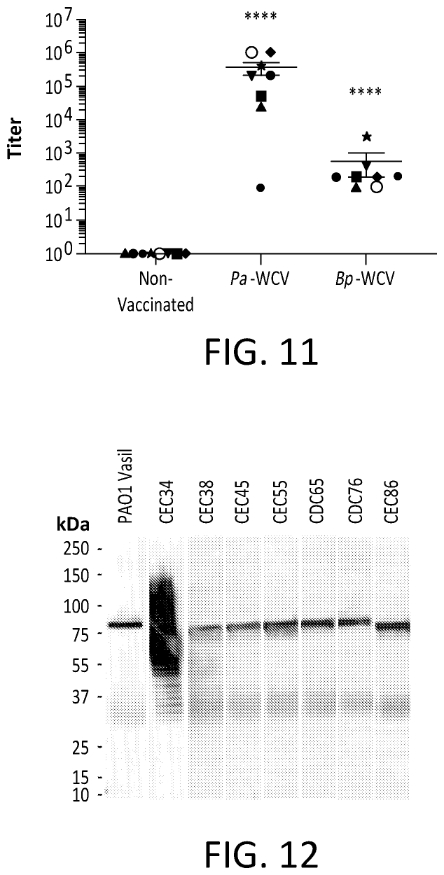

To explore whether antigens from B. pertussis could provide protection against clinical P. aeruginosa isolates, shown in Table 3, the sequence conservation of P. aeruginosa OprF and GroEL amongst 130 genome-sequenced clinical strains from CF patients was determined. OprF was 100% conserved across these isolates, and GroEL was 100% conserved in all but one clinical isolate, where the GroEL protein contained a single amino acid replacement (V525M). Binding of Bp-WCV serum to these isolates was then tested by ELISA. With ELISA, we observed that the serum from adjuvant-only vaccinated animals did not bind the clinical isolates, while antibodies in the pooled Pa-WCV serum bound each clinical P. aeruginosa isolates. Finally, there was consistent and significant binding of antibodies in the Bp-WCV serum to each P. aeruginosa clinical isolate tested, as shown in FIG. 11.

Subsequent western blot analysis of IgG binding to whole cell lysates indicated that antibodies present in the Bp-WCV serum retained binding to at least two antigens in each clinical isolate shown in Table 3, with molecular weights consistent with P. aeruginosa OprF and GroEL, as shown in FIG. 12. In the case of one clinical isolate (CEC34), antibodies in the Bp-WCV serum reacted with a much larger number of antigens.

The data indicate that antibodies raised in response to Bp-WVC vaccination recognizes antigens in a wide variety of clinical P. aeruginosa isolates, suggesting potential future applications for vaccine development.

### Example 9. Survival of Vaccinated Mice Challenged with a Lethal Dose of P. aeruginosa in a Sepsis Model

Five-week-old CD-1 mice were vaccinated with:
recombinant B. pertussis OmpA formulated with alum,
a whole cell P. aeruginosa vaccine (PaWCV), or
a whole cell B. pertussis vaccine (BpWCV).

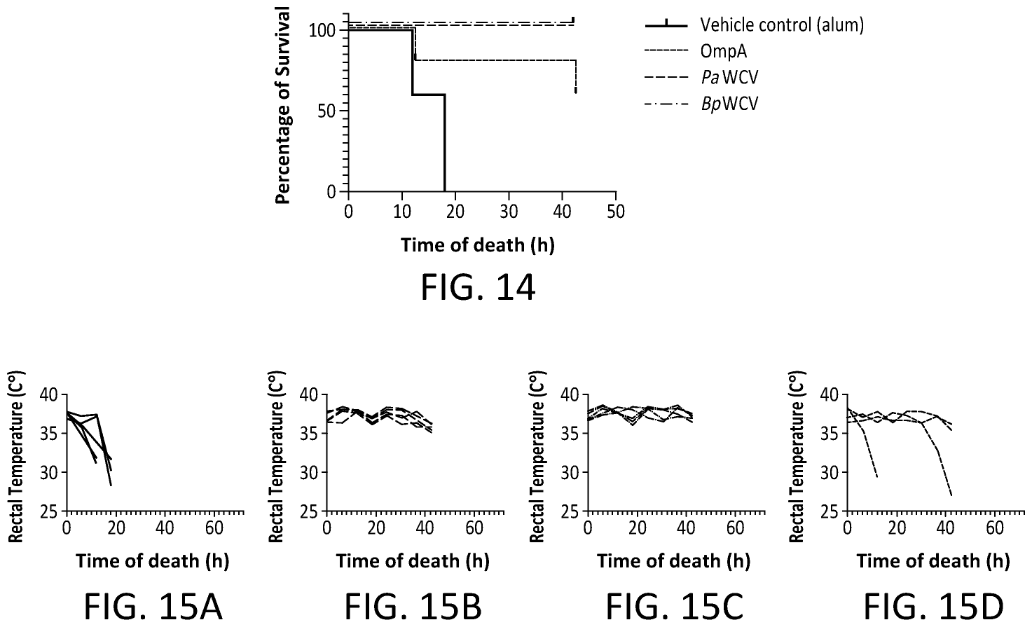

Alum alone was administered as vehicle control. Mice were vaccinated at day 0 and day 21 and challenged on day 35 with 5×10⁶ CFU of P. aeruginosa intraperitoneally (LD100). Survival was quantified over time. Results are shown in FIG. 14. Mice vaccinated with either BpWCV or PaWCV survived for over 43 hours following challenge with P. aeruginosa, indicating that a whole cell vaccine of either B. pertussis or P. aeruginosa is effective against challenge with P. aeruginosa.

Mice treated with alum alone had a survival rate of:
60% 12 hours after challenge with P. aeruginosa; and
0% 18 hours after challenge with P. aeruginosa.

Mice vaccinated with recombinant B. pertussis OmpA had a survival rate of:
80% 12 hours after challenge with P. aeruginosa; and
60% 48 hours after challenge with P. aeruginosa.

Thus, recombinant B. pertussis OmpA, used as a vaccine, increases the survival rate of CD-1 mice exposed to a lethal dose of P. aeruginosa.

### Example 10. Temperature Over Time of Vaccinated Mice Challenged with a Lethal Dose of P. aeruginosa in a Sepsis Model

Five-week-old CD-1 mice were vaccinated with:
recombinant B. pertussis OmpA formulated with alum,
a whole cell P. aeruginosa vaccine (PaWCV), or
a whole cell B. pertussis vaccine (BpWCV).

Alum alone was administered as vehicle control. Mice were vaccinated at day 0 and day 21 and challenged on day 35 with 5×10⁶ CFU of P. aeruginosa intraperitoneally (LD100). Rectal temperature was monitored over time. Results are shown in FIGS. 15A to 15D.

A first group of mice (n=5) vaccinated with BpWCV maintained a steady rectal temperature of 36° C. to 38° C. following challenge with P. aeruginosa (FIG. 15C). A second group of mice (n=5) vaccinated with PaWCV maintained a steady rectal temperature of 35° C. to 38° C. following challenge (FIG. 15B). This shows that a whole cell vaccine of either B. pertussis or P. aeruginosa is effective against challenge with P. aeruginosa.

A third group of mice (n=5) treated with alum alone showed a drastic decline in rectal temperature from about 37° C. to 38° C. immediately following challenge to about 28° C. to 32° C. prior to death (FIG. 15A).

A fourth group of mice (n=5) was vaccinated with recombinant B. pertussis OmpA (FIG. 15D). 80% of the fourth group of mice maintained a steady rectal temperature of 36° C. to 38° C. for at least 30 hours following challenge. 60% of the fourth group of mice maintained a steady rectal temperature of at least 35° C. for at least 42 hours following challenge.

### Example 11. Isolation of Cross-Reactive Antibodies Against B. pertussis Antigens

Five-week-old CD-1 mice will be injected with the following antigen peptides:
B. pertussis OmpA of SEQ ID NO: 8 (n=5),
a fragment of the B. pertussis OmpA including SEQ ID NO: 9 (n=5),
a fragment of the B. pertussis OmpA including SEQ ID NO: 12 (n=5),
a fragment of the B. pertussis OmpA including SEQ ID NO: 10 (n=5), or
a fragment of the B. pertussis OmpA including SEQ ID NO: 11 (n=5).
   The various antigens will each be conjugated to the carrier protein KLH. Mice will be vaccinated at day 0 and day 21, and serum will be screened for antibody titer against B. pertussis beginning on day 35. Once an adequate antibody titer builds up, antibodies will be recovered from serum and tested for cross-reactivity against P. aeruginosa OprF.

### Example 12. Isolation of Cross-Reactive Antibodies Against B. pertussis Antigens

Five-week-old CD-1 mice will be injected with the following antigen peptides:
B. pertussis GroEL having SEQ ID NO: 16 (n=5),
A B. pertussis GroEL fragment having amino acid residues 30 to 40 of SEQ ID NO: 16 (n=5),
A B. pertussis GroEL fragment having amino acid residues 50 to 98 of SEQ ID NO: 16 (n=5), A B. pertussis GroEL fragment having amino acid residues 168 to 178 of SEQ ID NO: 16 (n=5),
A B. pertussis GroEL fragment having amino acid residues 189 to 204 of SEQ ID NO: 16 (n=5),
A B. pertussis GroEL fragment having amino acid residues 251 to 304 of SEQ ID NO: 16 (n=5),
A B. pertussis GroEL fragment having amino acid residues 326 to 349 of SEQ ID NO: 16 (n=5), or
A B. pertussis GroEL fragment having amino acid residues 381 to 419 of SEQ ID NO: 16 (n=5).
   The various antigens will each be conjugated to the carrier protein KLH. Mice will be vaccinated at day 0 and day 21, and serum will be screened for antibody titer against B. pertussis beginning on day 35. Once an adequate antibody titer builds up, antibodies will be recovered from serum and tested for cross-reactivity against P. aeruginosa GroEL.

### Example 13. Isolation of Cross-Reactive Monoclonal Antibodies Against B. pertussis Antigens

Five-week-old CD-1 mice will be injected with the following antigen peptides:
B. pertussis OmpA of SEQ ID NO: 8 (n=5),
a fragment of the B. pertussis OmpA including SEQ ID NO: 9 (n=5),
a fragment of the B. pertussis OmpA including SEQ ID NO: 12 (n=5),
a fragment of the B. pertussis OmpA including SEQ ID NO: 10 (n=5), or
a fragment of the B. pertussis OmpA including SEQ ID NO: 11 (n=5).

The various antigens will each be conjugated to the carrier protein KLH. Mice will be vaccinated at day 0 and day 21, and serum will be screened for antibody titer against B. pertussis beginning on day 35. Once an adequate antibody titer builds up, the mice will be sacrificed. Antibody-secreting spleen cells from each group of the immunized mice will be fused with immortal myeloma cells to create monoclonal hybridoma cell lines that express the desired antibodies, e.g., antibodies against B. pertussis OmpA, antibodies against a fragment of the B. pertussis OmpA including SEQ ID NO: 9, etc. Each target antibody will be recovered from a respective monoclonal hybridoma cell line and tested for cross-reactivity against P. aeruginosa OprF.

### Example 14. Survival of Mice Vaccinated with B. pertussis GroEL Antibodies, when Challenged with P. aeruginosa

Five-week-old CD-1 mice will be vaccinated with:
an antibody against B. pertussis GroEL having SEQ ID NO: 16 (n=5), generated according to Example 12,
an antibody against a B. pertussis GroEL fragment having amino acid residues 30 to 40 of SEQ ID NO: 16 (n=5), generated according to Example 12,
an antibody against a B. pertussis GroEL fragment having amino acid residues 50 to 98 of SEQ ID NO: 16 (n=5), generated according to Example 12,
an antibody against a B. pertussis GroEL fragment having amino acid residues 168 to 178 of SEQ ID NO: 16 (n=5), generated according to Example 12,
an antibody against a B. pertussis GroEL fragment having amino acid residues 189 to 204 of SEQ ID NO: 16 (n=5), generated according to Example 12,
an antibody against a B. pertussis GroEL fragment having amino acid residues 251 to 304 of SEQ ID NO: 16 (n=5), generated according to Example 12,
an antibody against a B. pertussis GroEL fragment having amino acid residues 326 to 349 of SEQ ID NO: 16 (n=5), generated according to Example 12, or
an antibody against a B. pertussis GroEL fragment having amino acid residues 381 to 419 of SEQ ID NO: 16 (n=5), generated according to Example 12.

Mice will be vaccinated at day 0 and day 21 and will be challenged on day 35 with 5×10⁶ CFU of P. aeruginosa intraperitoneally (LD100). Survival will be quantified over time, as compared to control mice administered an alum control. B. pertussis GroEL antibodies which increase survival of vaccinated mice relative to an alum control will be assessed for potential therapeutic use against P. aeruginosa.

### Example 15. Survival of Mice Vaccinated with B. pertussis OmpA Antibodies, when Challenged with P. aeruginosa

Five-week-old CD-1 mice will be vaccinated with:
an antibody against B. pertussis OmpA of SEQ ID NO: 8 (n=5) generated according to Example 13,
an antibody against a fragment of the B. pertussis OmpA including SEQ ID NO: 9 (n=5) generated according to Example 13,
an antibody against a fragment of the B. pertussis OmpA including SEQ ID NO: 12 (n=5) generated according to Example 13,
an antibody against a fragment of the B. pertussis OmpA including SEQ ID NO: 10 (n=5) generated according to Example 13, or
an antibody against a fragment of the B. pertussis OmpA including SEQ ID NO: 11 (n=5) generated according to Example 13.

Mice will be vaccinated at day 0 and day 21 and will be challenged on day 35 with 5×10⁶ CFU of P. aeruginosa intraperitoneally (LD100). Survival will be quantified over time, as compared to control mice administered an alum control. B. pertussis OmpA antibodies which increase survival of vaccinated mice relative to an alum control will be assessed for potential therapeutic use against P. aeruginosa.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method of immunizing a mammalian patient against infection by a bacterial pathogen, comprising administering a pertussis antigen to the mammalian patient,
wherein the pertussis antigen is least one of:
a chaperonin protein GroEL from Bordetella pertussis, or a fragment thereof;
an OmpA protein of Bordetella pertussis, or a fragment thereof; and
wherein the bacterial pathogen expresses a protein having at least 45% identity to the pertussis antigen.

2. The method of claim 1, wherein the bacterial pathogen is:
a bacteria from a genus selected from the group consisting of a genus Bordetella, a genus Pseudomonas, a genus Escherichia, a genus Enterococcus, a genus Staphylococcus, a genus Klebsiella, a genus Acinetobacter, and a genus Enterobacter;
an ESKAPE pathogen selected from the group consisting of Enterococcus faecium, Staphylococcus aureus, Klebsiella pneumoniae, Acinetobacter baumannii, Pseudomonas aeruginosa, and an Enterobacter species; or
a combination thereof.

3. The method of claim 1, wherein the pertussis antigen is conjugated to a carrier protein or an amino acid tag.

4. The method of claim 1, wherein the pertussis antigen is, and the bacterial pathogen expresses a protein having at least 60% identity to the chaperonin protein GroEL from Bordetella pertussis.

5. The method of claim 1, wherein:
the bacterial pathogen expresses a protein having at least 60% identity to the chaperonin protein GroEL from Bordetella pertussis; and
the pertussis antigen is:
the chaperonin protein GroEL from Bordetella pertussis; or
a fragment of the chaperonin protein GroEL from Bordetella pertussis having at least 85% identity to:
a sequence having amino acid residues 30 to 40 of SEQ ID NO: 16,
a sequence having amino acid residues 50 to 98 of SEQ ID NO: 16,
a sequence having amino acid residues 168 to 178 of SEQ ID NO: 16,
a sequence having amino acid residues 189 to 204 of SEQ ID NO: 16,
a sequence having amino acid residues 251 to 304 of SEQ ID NO: 16,
a sequence having residues 326 to 349 of SEQ ID NO: 16; or
a sequence having amino acid residues 381 to 419 of SEQ ID NO: 16.

6. The method of claim 1, wherein:
the pertussis antigen is a fragment of the OmpA protein of Bordetella pertussis comprising SEQ ID NO: 9, and the bacterial pathogen expresses a protein having a sequence with at least 50% identity to SEQ ID NO: 9;
the pertussis antigen is a fragment of the OmpA protein of Bordetella pertussis comprising SEQ ID NO: 12, and the bacterial pathogen expresses a protein having a sequence with at least 80% identity to SEQ ID NO: 12;
the pertussis antigen is a fragment of the OmpA protein of Bordetella pertussis comprising SEQ ID NO: 10, and the bacterial pathogen expresses a protein having a sequence with at least 90% identity to SEQ ID NO: 10; or
the pertussis antigen is a fragment of the OmpA protein of Bordetella pertussis comprising SEQ ID NO: 11, and the bacterial pathogen expresses a protein having a sequence with at least 90% identity to SEQ ID NO: 11.

7. The method of claim 1, wherein the pertussis antigen is administered intranasally, intravenously, intramuscularly, subcutaneously, intradermally, orally, rectally, or intraperitoneally.

8. The method of claim 1, wherein the pertussis antigen is administered in combination with an adjuvant selected from the group consisting of curdlan, alum, amorphous aluminum hydroxyphosphate sulfate, aluminum hydroxide, aluminum phosphate, monophosphoryl lipid A, squalene, cytosine phosphoguanine, MF59, AS03, AS04, BECC adjuvants (Bacterial Enzymatic Combinatorial Chemistry), SWE, and combinations thereof.

9. The method of claim 1, wherein:
the pertussis antigen is administered to the mammalian patient in combination with a second antigen to the bacterial pathogen; and
the bacterial pathogen is selected from the group consisting of bacteria from a genus Pseudomonas, a genus Escherichia, a genus Enterococcus, a genus Staphylococcus, a genus Klebsiella, a genus Acinetobacter, and a genus Enterobacter.

10. The method of claim 9, wherein the pertussis antigen is administered in combination with an adjuvant selected from the group consisting of curdlan, alum, amorphous aluminum hydroxyphosphate sulfate, aluminum hydroxide, aluminum phosphate, monophosphoryl lipid A, squalene, cytosine phosphoguanine, MF59, AS03, AS04, BECC (Bacterial Enzymatic Combinatorial Chemistry) adjuvants, SWE, and combinations thereof.

11. A method of immunizing a mammalian patient against infection by a bacterial pathogen, comprising administering a Pseudomonas antigen to the mammalian patient,
wherein the Pseudomonas antigen is least one of:
a chaperonin protein GroEL from Pseudomonas aeruginosa, or a fragment thereof;
an OprF protein from Pseudomonas aeruginosa, or a fragment thereof; and
wherein:
the bacterial pathogen expresses a protein having at least 45% identity to the Pseudomonas antigen.

12. The method of claim 11, wherein the bacterial pathogen is selected from the group consisting of bacteria from a genus Bordetella, a genus Escherichia, a genus Enterococcus, a genus Staphylococcus, a genus Klebsiella, a genus Acinetobacter, and a genus Enterobacter.

13. The method of claim 11, wherein:
the Pseudomonas antigen is the chaperonin protein GroEL from Pseudomonas aeruginosa, and the bacterial pathogen expresses a protein having at least 60% identity to the chaperonin protein GroEL from Pseudomonas aeruginosa; or
the Pseudomonas antigen is a fragment of the OprF protein of Pseudomonas aeruginosa comprising an OmpA domain, and the bacterial pathogen expresses a protein having a sequence with at least 50% identity to the OmpA domain.

14. The method of claim 11, wherein:
the Pseudomonas antigen is a fragment of the OprF protein of Pseudomonas aeruginosa comprising SEQ ID NO: 3, and the bacterial pathogen expresses a protein having a sequence with at least 50% identity to SEQ ID NO: 3;
the Pseudomonas antigen is a fragment of the OprF protein of Pseudomonas aeruginosa comprising SEQ ID NO: 6, and the bacterial pathogen expresses a protein having a sequence with at least 80% identity to SEQ ID NO: 6;
the Pseudomonas antigen is a fragment of the OprF protein of Pseudomonas aeruginosa comprising SEQ ID NO: 4, and the bacterial pathogen expresses a protein having a sequence with at least 90% identity to SEQ ID NO: 4; or
the Pseudomonas antigen is a fragment of the OprF protein of Pseudomonas aeruginosa comprising SEQ ID NO: 5, and the bacterial pathogen expresses a protein having a sequence with at least 90% identity to SEQ ID NO: 5.

15. The method of claim 11, wherein the Pseudomonas antigen is administered intranasally, intravenously, intramuscularly, subcutaneously, intradermally, orally, rectally, or intraperitoneally.

16. The method of claim 11, wherein the Pseudomonas antigen is administered in combination with an adjuvant selected from the group consisting of curdlan, alum, amorphous aluminum hydroxyphosphate sulfate, aluminum hydroxide, aluminum phosphate, monophosphoryl lipid A, squalene, cytosine phosphoguanine, and combinations thereof.

17. A therapeutically effective antibody, wherein the antibody is selected from the group consisting of:
an antibody generated against B. pertussis GroEL having at least 85% identity to SEQ ID NO: 16 or a fragment thereof, wherein the antibody generated against B. pertussis GroEL has cross reactivity against P. aeruginosa bacteria expressing P. aeruginosa GroEL;
an antibody generated against P. aeruginosa GroEL having at least 85% identity to SEQ ID NO: 14 or a fragment thereof, wherein the antibody generated against P. aeruginosa GroEL has cross reactivity against other species of bacteria expressing GroEL; and
an antibody generated against B. pertussis OmpA having at least 85% identity to SEQ ID NO: 8 or a fragment thereof, wherein the antibody generated against B. pertussis OmpA has cross reactivity against other species of bacteria.

18. The therapeutically effective antibody of claim 17, wherein the antibody is generated against the fragment of the B. pertussis GroEL,
where the fragment of B. pertussis GroEL is:
A B. pertussis GroEL fragment having at least 85% identity to the sequence having amino acid residues 30 to 40 of SEQ ID NO: 16;
A B. pertussis GroEL fragment having at least 85% identity to the sequence having amino acid residues 50 to 98 of SEQ ID NO: 16;
A B. pertussis GroEL fragment having at least 85% identity to the sequence having amino acid residues 168 to 178 of SEQ ID NO: 16;
A B. pertussis GroEL fragment having at least 85% identity to the sequence having amino acid residues 189 to 204 of SEQ ID NO: 16;
A B. pertussis GroEL fragment having at least 85% identity to the sequence having amino acid residues 251 to 304 of SEQ ID NO: 16;
A B. pertussis GroEL fragment having at least 85% identity to the sequence having amino acid residues 326 to 349 of SEQ ID NO: 16; or
A B. pertussis GroEL fragment having at least 85% identity to the sequence having amino acid residues 381 to 419 of SEQ ID NO: 16.

19. The therapeutically effective antibody of claim 17, wherein the antibody is generated against the fragment of the P. aeruginosa GroEL,
wherein the fragment of P. aeruginosa GroEL is:
a P. aeruginosa GroEL fragment having at least 85% identity to the sequence having amino acid residues 30 to 40 of SEQ ID NO: 14;
a P. aeruginosa GroEL fragment having at least 85% identity to the sequence having amino acid residues 50 to 98 of SEQ ID NO: 14;
a P. aeruginosa GroEL fragment having at least 85% identity to the sequence having amino acid residues 168 to 178 of SEQ ID NO: 14;
a P. aeruginosa GroEL fragment having at least 85% identity to the sequence having amino acid residues 189 to 204 of SEQ ID NO: 14;
a P. aeruginosa GroEL fragment having at least 85% identity to the sequence having amino acid residues 251 to 304 of SEQ ID NO: 14;
a P. aeruginosa GroEL fragment having at least 85% identity to the sequence having amino acid residues 326 to 349 of SEQ ID NO: 14; or
a P. aeruginosa GroEL fragment having at least 85% identity to the sequence having amino acid residues 381 to 419 of SEQ ID NO: 14.

20. The therapeutically effective antibody of claim 17, wherein the antibody is generated against the fragment of B. pertussis OmpA having SEQ ID NO: 8,
where the fragment of B. pertussis OmpA is:
a fragment of the B. pertussis OmpA having at least 85% identity to SEQ ID NO: 9,
a fragment of the B. pertussis OmpA having at least 85% identity to SEQ ID NO: 12,
a fragment of the B. pertussis OmpA having at least 85% identity to SEQ ID NO: 10, or
a fragment of the B. pertussis OmpA having at least 85% identity to SEQ ID NO: 11.


SEQUENCE LISTING (17 sequences)

SEQ ID NO: 1 - DNA, 1053 bp, Pseudomonas aeruginosa
  source 1..1053: mol_type = unassigned DNA
atgaaactgaagaacaccttaggcgttgtcatcggctcgctggttgccgcttcggcaatgaacgcctttgcccagggccagaactcggtagagatcgaagccttcggcaagcgctacttcaccgacagcgttcgcaacatgaagaacgcggacctgtacggcggctcgatcggttacttcctgaccgacgacgtcgagctggcgctgtcctacggtgagtaccatgacgttcgtggcacctacgaaaccggcaacaagaaggtccacggcaacctgacctccctggacgccatctaccacttcggtaccccgggcgtaggtctgcgtccgtacgtgtcggctggtctggctcaccagaacatcaccaacatcaacagcgacagccaaggccgtcagcagatgaccatggccaacatcggcgctggtctgaagtactacttcaccgagaacttcttcgccaaggccagcctcgacggccagtacggtctggagaagcgtgacaacggtcaccagggcgagtggatggctggcctgggcgtcggcttcaacttcggtggttcgaaagccgctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaagccgaagtagaagctgaagccaagtaa

SEQ ID NO: 2 - protein, 350 aa, Pseudomonas aeruginosa
MKLKNTLGVVIGSLVAASAMNAFAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 3 - protein, 120 aa, Pseudomonas aeruginosa
AVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 4 - protein, 24 aa, Pseudomonas aeruginosa
GHTDSVGTDAYNQKLSERRANAVR

SEQ ID NO: 5 - protein, 17 aa, Pseudomonas aeruginosa
DNATAEGRAINRRVEAE

SEQ ID NO: 6 - protein, 67 aa, Pseudomonas aeruginosa
GHTDSVGTDAYNQKLSERRANAVRDVLVNEYGVEGGRVNAVGYGESRPVADNATAEGRAINRRVEAE

SEQ ID NO: 7 - DNA, 582 bp, Bordetella pertussis
  source 1..582: mol_type = unassigned DNA
atgaacaaaccctccaaattcgctctggcgctcgccttcgccgctgttacggcctctggtgcagcttccgcgcaaaccgtggacaactggcgcaatccgtttggcgacgtttggaagaacggcaccaatgaactgtgctggcgcgatgcgttctggaccccggctaccggcatccccggttgcgacggcgttccggtcgctcagaaggaaaagtccgctcccatggccgccaaggtcgtgttcaatgctgacaccttcttcgacttcgacaagtcgacgctgaagccggaaggccgccagctgctggatcaagtcgcccagcaagccggcacgatcgatctggaaacgatcatcgccgttggccacacggactcgatcggcaccgaagcctacaaccagaagctgtccgagcgccgtgccgctgcggtcaagacctacctggtcagcaagggtatcgaccccaaccgtatctacacggaaggcaagggcgaactgcaaccgatcgcttcgaacaagacgcgtgaaggccgtgcccagaaccgtcgcgtggaaatcgaaatcgtcggtagccgcaagaactaa

SEQ ID NO: 8 - protein, 193 aa, Bordetella pertussis
MNKPSKFALALAFAAVTASGAASAQTVDNWRNPFGDVWKNGTNELCWRDAFWTPATGIPGCDGVPVAQKEKSAPMAAKVVFNADTFFDFDKSTLKPEGRQLLDQVAQQAGTIDLETIIAVGHTDSIGTEAYNQKLSERRAAAVKTYLVSKGIDPNRIYTEGKGELQPIASNKTREGRAQNRRVEIEIVGSRKN

SEQ ID NO: 9 - protein, 120 aa, Bordetella pertussis
PMAAKVVFNADTFFDFDKSTLKPEGRQLLDQVAQQAGTIDLETIIAVGHTDSIGTEAYNQKLSERRAAAVKTYLVSKGIDPNRIYTEGKGELQPIASNKTREGRAQNRRVEIEIVGSRKN

SEQ ID NO: 10 - protein, 25 aa, Bordetella pertussis
GHTDSIGTEAYNQKLSERRAAAVKT

SEQ ID NO: 11 - protein, 17 aa, Bordetella pertussis
SNKTREGRAQNRRVEIE

SEQ ID NO: 12 - protein, 66 aa, Bordetella pertussis
GHTDSIGTEAYNQKLSERRAAAVKTYLVSKGIDPNRIYTEGKGELQPIASNKTREGRAQNRRVEIE

SEQ ID NO: 13 - DNA, 1644 bp, Pseudomonas aeruginosa
  source 1..1644: mol_type = unassigned DNA
atggctgccaaagaagttaagttcggcgattccgctcgcaagaaaatgctggtcggcgtgaacgtgctggccgatgccgtcaaggccaccctcggcccgaaaggccgcaacgtggttctggacaagtccttcggcgctccgaccatcaccaaggacggcgtttccgtcgccaaggaaatcgagctgaaagacaagttcgagaacatgggcgcgcaactggtgaaagacgttgcctccaaggccaacgacgctgccggtgacggcaccaccaccgcgaccgtcctggcccaggccatcgtcaacgaaggcctgaaggccgttgccgccggcatgaacccgatggacctgaagcgtggcatcgacaaggccaccgtggctatcgtcgcccagctgaaagagctggccaagccctgcgccgacaccaaggccatcgcccaggtaggcaccatctccgccaactccgacgagtccatcggccagatcattgccgaagccatggaaaaagtcggtaaagaaggcgtgatcaccgtcgaggaaggctcgggcctggaaaacgaactgtccgtcgtcgaaggcatgcagttcgatcgcggctacctgtccccctacttcgtgaacaagccggacaccatggctgccgagctggatagcccgctgctgctgctggtcgacaagaagatctccaacatccgcgaaatgctgccggtgctggaagccgtcgccaaggccggccgtccgctgctgatcgtcgccgaggacgtcgagggcgaagccctggccaccctggtggtgaacaacatgcgtggcatcgtcaaggtcgcggctgtcaaggctccgggcttcggcgatcgccgcaaggccatgctgcaggacatcgccatcctcaccggcggtaccgtgatcagcgaagaagtcggcctgagcctggaaggcgctaccctggagcacctgggcaacgccaagcgcgtcgtgatcaacaaggaaaacaccaccatcatcgatggcgccggtgtgcaggctgatatcgaagcccgcgtcctgcagatccgcaagcagatcgaggaaaccacttccgactacgaccgcgagaagctgcaagagcgcctggccaagctggccggcggtgttgccgtgatcaaggtaggcgctgccaccgaagtcgagatgaaagagaagaaagcccgcgtcgaagacgccctgcacgctacccgtgcagcggtggaagagggcgtggttcccggcggcggcgtagccctggttcgtgccctgcaagccatcgaaggcctgaagggtgacaacgaggagcagaacgtcggtatcgccctgctgcgtcgcgccgtcgaatcgccgctgcgccagatcgtggccaacgccggcgacgagccgagcgtagtggtcgacaaggtcaagcagggttccggcaactacggcttcaacgctgctaccggcgtgtacggcgacatgatcgagatgggcatcctggacccggccaaggtcactcgttccgctctgcaggctgcggcctccatcggcggtctgatgatcaccaccgaagccatggttgccgagatcgtggaagacaagccggccatgggcggcatgcctgacatgggcggcatgggcggcatgggcggcatgatgtaa

SEQ ID NO: 14 - protein, 547 aa, Pseudomonas aeruginosa
MAAKEVKFGDSARKKMLVGVNVLADAVKATLGPKGRNVVLDKSFGAPTITKDGVSVAKEIELKDKFENMGAQLVKDVASKANDAAGDGTTTATVLAQAIVNEGLKAVAAGMNPMDLKRGIDKATVAIVAQLKELAKPCADTKAIAQVGTISANSDESIGQIIAEAMEKVGKEGVITVEEGSGLENELSVVEGMQFDRGYLSPYFVNKPDTMAAELDSPLLLLVDKKISNIREMLPVLEAVAKAGRPLLIVAEDVEGEALATLVVNNMRGIVKVAAVKAPGFGDRRKAMLQDIAILTGGTVISEEVGLSLEGATLEHLGNAKRVVINKENTTIIDGAGVQADIEARVLQIRKQIEETTSDYDREKLQERLAKLAGGVAVIKVGAATEVEMKEKKARVEDALHATRAAVEEGVVPGGGVALVRALQAIEGLKGDNEEQNVGIALLRRAVESPLRQIVANAGDEPSVVVDKVKQGSGNYGFNAATGVYGDMIEMGILDPAKVTRSALQAAASIGGLMITTEAMVAEIVEDKPAMGGMPDMGGMGGMGGMM

SEQ ID NO: 15 - DNA, 1644 bp, Bordetella pertussis
  source 1..1644: mol_type = unassigned DNA
atggctgccaagcaagttctgtttgccgacgaagcccgcgtgcgcatcgtgcgcggcgtgaacgtcctcgccaacgccgtcaagaccaccctgggccccaagggtcgcaacgtcgtgctggagcgctcgttcggcgccccgacggtgaccaaggacggcgtgtccgtcgccaaggaaatcgaactgaaggacaagttcgagaacatcggcgcccagctggtcaaggacgttgcctccaagacctccgacaacgccggtgacggcaccaccaccgccaccgtgctggcccaggccgtcgttcaggaaggcctgaagtacgtcgccgccggcttcaacccgatcgacctcaaacgcggcatcgacaaggccgtggccgccgccgtggaagagctgaagaaactgtccaagccggtcaccaccagcaaggaaatcgcccaggttggctcgatctcggccaacagcgatgcctcgatcggccagatcatcgctgacgcgatggacaaggtcggcaaggaaggcgtgatcaccgtcgaagacggcaagtcgctggaaaacgaactggatgtcgtcgaaggcatgcaattcgaccgcggctacctgtcgccctacttcatcaacagcccggaaaagcaagtcgccgcgctggacgatccgtatgtcctgatctacgacaagaaggtcagcaacatccgtgacctgctgcccgtgctggagcaagtcgccaagtcgagccgtccgctgctgatcatcgccgaagacgtcgaaggcgaagcgctggccaccctggtcgtgaacaacatccgcggcatcctgaagaccaccgccgtcaaggccccgggcttcggcgaccgtcgcaaggccatgctggaagacatcgccatcctgacgggcggcacggtgatctccgaagaaaccggcatgtcgctggaaaaggccacgctgcaggatctgggccaggccaagcgcatcgaagtggccaaggaaaacaccacgatcatcgacggcgccggcgacggcaagtcgatcgaagcgcgcgtcaagcagatccgcgcccagatcgaggaagccacctcggactacgaccgcgagaagctgcaagagcgcgtggccaagctggccggcggcgttgccgtgattcgcgtcggtgctgccaccgaagtcgaaatgaaggaaaagaaggctcgcgtcgaagacgccctgcacgccacgcgtgccgcggtggaagaaggcgtggttcctggcggtggcgttgcgctgctgcgcgccaagcaggccatcactggcctgaagggcgacacggctgaccagaacgccggcatcaagctgatcctgcgcgctgtcgaagagcccctgcgcaccatcgtcaccaacgccggcgacgaagccagcgtggtggtcaacaccgtgctcaacggcaagggcaactacggctacaacgccgcgaccggcgagtacggcgacctggtcgagcaaggcgtgctggacccgaccaaggtcacccgcaccgcgctgcagaacgccgcttcggtggccagcctgctgctgacggccgaagccgccgtggtcgagctgatggaaaacaagcccgctgctgctccggcaatgcccggcggcatgggcggcatgggcggcatggacttctaa

SEQ ID NO: 16 - protein, 547 aa, Bordetella pertussis
MAAKQVLFADEARVRIVRGVNVLANAVKTTLGPKGRNVVLERSFGAPTVTKDGVSVAKEIELKDKFENIGAQLVKDVASKTSDNAGDGTTTATVLAQAVVQEGLKYVAAGFNPIDLKRGIDKAVAAAVEELKKLSKPVTTSKEIAQVGSISANSDASIGQIIADAMDKVGKEGVITVEDGKSLENELDVVEGMQFDRGYLSPYFINSPEKQVAALDDPYVLIYDKKVSNIRDLLPVLEQVAKSSRPLLIIAEDVEGEALATLVVNNIRGILKTTAVKAPGFGDRRKAMLEDIAILTGGTVISEETGMSLEKATLQDLGQAKRIEVAKENTTIIDGAGDGKSIEARVKQIRAQIEEATSDYDREKLQERVAKLAGGVAVIRVGAATEVEMKEKKARVEDALHATRAAVEEGVVPGGGVALLRAKQAITGLKGDTADQNAGIKLILRAVEEPLRTIVTNAGDEASVVVNTVLNGKGNYGYNAATGEYGDLVEQGVLDPTKVTRTALQNAASVASLLLTAEAAVVELMENKPAAAPAMPGGMGGMGGMDF

SEQ ID NO: 17 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM